(19) United States
(12) Patent Application Publication
(10) Pub. No.: US 2012/0289432 A1
(43) Pub. Date: Nov. 15, 2012

(54) PI3K MODULATORS, RHO KINASE MODULATORS AND METHODS OF IDENTIFYING AND USING SAME

(75) Inventors: Huayun Deng, Painted Post, NY (US); Ye Fang, Painted Post, NY (US); Ann MeeJin Ferrie, Painted Post, NY (US); Haiyan Sun, Chandler, AZ (US); Elizabeth Tran, Painted Post, NY (US)

(73) Assignee: Corning Incorporated, Corning, NY (US)

(21) Appl. No.: 13/519,271

(22) PCT Filed: Dec. 16, 2010
(86) PCT No.: PCT/US10/60648

§ 371 Date: Jun. 26, 2012

Related U.S. Application Data
(60) Provisional application No. 61/291,736, filed on Dec. 31, 2009.

(51) Int. Cl. C12Q 1/48; C40B 30/06; G01N 21/41

(52) U.S. Cl. 506/10; 435/15

(57) ABSTRACT

Disclosed are methods to characterize PI3K inhibitors and Rho kinase inhibitors using label-free cellular assays. Disclosed are also methods to characterize a cell whether it has a deregulated PI3K pathway or not.

### CROSS-REFERENCE TO RELATED APPLICATIONS

This application claims the benefit of priority under 35 U.S.C. §119(e) of U.S. Provisional Application Ser. No. 61/291,736 filed on Dec. 31, 2009.

### BACKGROUND

Phosphoinositide 3-kinase (PI3K) pathway is a crucial signaling cascade involved in various cellular processes. The PI3K pathway regulates many physiological functions including cell growth, survival, amplification and apoptosis. PI3Ks are dual lipid and protein kinases that are activated by a wide range of receptors including receptor tyrosine kinases (RTKs) and G protein-coupled receptors (GPCRs). PI3Ks catalyze the synthesis of the phosphatidylinositol (PI) second messengers PI(3)P, PI(3,4)P₂, and PI(3,4,5)P₃ (PIP₃). These phosphorylated lipids are produced at cellular membranes during signaling events and contribute to the recruitment and activation of various signaling components. In the appropriate cellular context, these lipids control diverse physiological processes including cell growth, survival, differentiation, and chemotaxis, via interactions with various signaling proteins including protein serine-threonine kinases, protein tyrosine kinases, and exchange factors that regulate heterotrimeric guanosine triphosphate (GTP)-binding proteins (G proteins). These proteins are located in the cytosol of unstimulated cells but, in response to lipid phosphorylation, accumulate at the plasma membrane because of their ability to associate with the newly formed phosphoinositides. At the membrane, these proteins become activated and initiate various local responses, including polymerization of actin, assembly of signaling complexes, and priming of protein kinase cascades. Hyperactivation of this pathway contributes to human cancers and defects in the pathway contribute to type II diabetes. The PI3K pathway is implicated in human diseases including diabetes and cancer, and understanding the intricacies of this pathway may provide new avenues for therapeutic intervention.

Rho GTPases represent a subset of the larger Ras superfamily and consist of over 20 intracellular signaling proteins. They are often referred to as the small GTPases because of their ˜20 kD size, with the most thoroughly characterized members being RhoA, Cdc42, and Rac1. They are ubiquitously expressed proteins known to be molecular switches for the transduction of signals from external stimuli through the activation of integrins, growth factor receptors, ion channels, and G-protein coupled receptors. The activation of the GTPase by receptor or non-receptor dependent mechanisms mediates the transition between an active GTP bound state and an inactive GDP bound state. Numerous studies have demonstrated the important roles of Rho GTPases in the regulation of gene transcription, cell proliferation, migration, cell division, and cell shape change. Thus, identification of Rho kinase inhibitor could lead to new drugs. Disclosed herein are methods to screen for Rho kinase inhibitors.

### SUMMARY

Disclosed herein are methods to screen phosphoinositide 3-kinase (PI3K) inhibitors using label-free biosensor cellular assays. For example, three methods are disclosed and their combinations to screen PI3K inhibitors—(1) molecules are screened with a label-free biosensor cellular assays against a PI3K inhibitor in a PI3K pathway-amplified cell; (2) molecules are screened with a label-free biosensor cellular assay against a marker whose biosensor cellular response contains PI3K pathway-associated contributions in a PI3K pathway-amplified cell; and (3) molecules are assayed with a label-free biosensor cellular assays against panels of markers, each panel of markers for a cell type, whose biosensor index, particularly biosensor DMR index, is used as an indicator for the compounds being PI3K inhibitors. Particularly, the disclosed methods separate Rho kinase (ROCK) inhibitors from PI3k inhibitors. The disclosed methods also characterize PI3K pathway deregulated cells.

Disclosed herein are methods to screen Rho kinase(ROCK) inhibitors using label-free biosensor cellular assays. The disclosed methods include three distinct methods and their combinations to screen ROCK inhibitors—(1) molecules are screened with a label-free biosensor cellular assays against a Rho kinase inhibitor in a Rho kinase inhibitor responsive cell; (2) molecules are screened with a label-free biosensor cellular assays against a marker whose biosensor cellular response contains Rho kinase-associated pathway contributions in a Rho kinase inhibitor responsive cell; and (3) molecules are assayed with a label-free biosensor cellular assays against panels of markers, each panel of markers for a cell type, whose biosensor index, particularly biosensor DMR index, is used as an indicator for the compounds being Rho kinase inhibitors.

### BRIEF DESCRIPTION OF THE DRAWINGS

FIG. 1 shows a flow chart for methods to screen PI3K inhibitors

FIG. 2 shows a flow chart for methods to screen PI3K inhibitors.

FIG. 3 shows a flow chart for methods to identify phenotypic and poly-pharmacology of aPI3K inhibitor.

FIG. 4A-4B shows the optical biosensor DMR signals of the human lung cancer cell line A549 in response to a well-known and reversible PI3K inhibitor LY294002 at different doses. (A) The real time kinetic responses of A549 cells upon stimulation with LY294002 at different doses. (B) The amplitudes of the LY294002 induced N-DMR event as a function of LY294002 doses. The amplitudes were measured at a time point of ˜50 min after stimulation with LY294002.

FIG. 5A-5B shows the optical biosensor DMR signals of the human lung cancer cell line A549 in response to a well-known and irreversible PI3K inhibitor wortmannin at different doses. (A) The real time kinetic responses of A549 cells upon stimulation with wortmannin at different doses. (B) The amplitudes of the wortmannin induced N-DMR event as a function of LY294002 doses. The amplitudes were measured at time point of ˜50 min after stimulation with wortmannin.

FIG. 6A-6D shows a specific panel of cells/markers-based biosensor cellular profiling method to determine the compounds to be PI3K kinase inhibitors. (A) The EGF-induced DMR signals in quiescent A431 cells in the absence (“buffer”) and presence of two PI3K inhibitors, quercetin and LY294002. The EGF concentration was 32 nM. (B) The histamine-induced DMR signals in A549 cells in the absence (“buffer”) and presence of two PI3K inhibitors, quercetin and LY294002. The histamine concentration was 10 micromolar. (C) The forskolin-induced DMR signals in A549 cells in the absence (“buffer”) and presence of two PI3K inhibitors, quercetin and LY294002. The forskolin concentration was 16 micromolar. (D) The ODN2006-induced DMR signals in HepG2 cells in the absence (“buffer”) and presence of two PI3K inhibitors, quercetin and LY294002. The ODN2006 concentration was 500 nM. In all experiments, the PI3K inhibitor concentration was 10 micromolar. At least four replicates were obtained. The error bars in all curves represent the standard deviation of the four replicates. EGF, histamine, forskolin, and ODN2006 were considered as markers, whose corresponding DMR signals all contain contributions from the PI3K pathway downstream from their corresponding targets.

FIG. 7A-7C shows a specific panel of cells/markers-based biosensor cellular profiling method to determine the compounds to be PI3K inhibitors. (A) The DMR signal of the known PI3K inhibitor LY294002 in quiescent A431 cells; (B) The DMR signal of the known PI3K inhibitor LY294002 in A549 cells; (C) The DMR modulation index of the known PI3K inhibitor LY294002 against panels of markers across the two cell lines (pinacidil, forskolin, and histamine for A549 cells, and EGF for A431 cells). The index was generated after normalizing each marker-induced DMR signal in the presence of the inhibitor to its corresponding signal in the absence of the inhibitor. The percentage of modulation for each marker by the inhibitor was plotted as a function of the marker. As shown from left to right in a sequential order, the DMR events used for the basis of the modulation percentage calculation are: the N-DMR (50 min after stimulation) for pinacidil in A549, the P-DMR (50 min after stimulation) for forskolin in A549, the early P-DMR event (˜10 min after histamine stimulation) and the late P-DMR event (˜30 min after histamine stimulation) for histamine in A549, and the early P-DMR event (˜5 min after EGF stimulation) and the subsequent N-DMR event (˜30 min after EGF stimulation) for EGF in A431 cells. In all cases, the amplitudes of respective DMR events were used as the basis to calculate the percentages of modulation by the PI3K inhibitors. The same procedure was used to generate FIGS. 8 to 14.

FIG. 8A-8C shows a specific panel of cells/markers-based biosensor cellular profiling method to determine the compounds to be PI3K inhibitors. (A) The DMR signal of the known PI3K inhibitor quercetin in the quiescent A431 cell line; (B) The DMR signal of the known PI3K inhibitor quercetin in the lung cancer cell line, A549; (C) The DMR modulation index of the known PI3K inhibitor quercetin against panels of markers across the two cell lines (pinacidil, forskolin, and histamine for A549 cells, and EGF for A431 cells).

FIG. 9A-9C shows a specific panel of cells/markers-based biosensor cellular profiling method to determine the compounds to be PI3K inhibitors. (A) The DMR signal of the known PI3Kγ inhibitor 5-Quinoxalin-6-ylmethylene-thiazolindine-2,4-dione in the quiescent A431 cells; (B) The DMR signal of the known PI3Kγ inhibitor 5-Quinoxalin-6-ylmethylene-thiazolindine-2,4-dione in lung cancer cell line A549; (C) The DMR modulation index of the known PI3Kγ inhibitor 5-Quinoxalin-6-ylmethylene-thiazolindine-2,4-dione against panels of markers across the two cell lines (pinacidil, forskolin, and histamine for A549 cells, and EGF for A431 cells).

FIG. 10A-10C shows a specific panel of cells/markers-based biosensor cellular profiling method to determine the compounds to be PI3K inhibitors. (A) The DMR signal of the known PI3Kβ inhibitor II (+/−)-7-Methyl-2-(morpholin-4-yl)-9-(1-phenylaminoethyl)-pyridol[1,2-a]-pyrimidin-4-one in the quiescent A431 cells; (B) The DMR signal of the known PI3Kβ inhibitor II (+/−)-7-Methyl-2-(morpholin-4-yl)-9-(1-phenylaminoethyl)-pyridol[1,2-a]-pyrimidin-4-one in lung cancer cell line A549; (C) The DMR modulation index of the known PI3Kβ inhibitor II (+/−)-7-Methyl-2-(morpholin-4-yl)-9-(1-phenylaminoethyl)-pyridol[1,2-a]-pyrimidin-4-one against panels of markers across the two cell lines (pinacidil, forskolin, and histamine for A549 cells, and EGF for A431 cells).

FIG. 11A-11C shows a specific panel of cells/markers-based biosensor cellular profiling method to determine the compounds to be PI3K inhibitors. (A) The DMR signal of the known PI3K inhibitor PI-103 in the quiescent A431 cells; (B) The DMR signal of the known PI3K inhibitor PI-103 in lung cancer cell line A549; (C) The DMR modulation index of the known PI3K inhibitor PI-103 against panels of markers across the two cell lines (pinacidil, forskolin, and histamine for A549 cells, and EGF for A431 cells).

FIG. 12A-12C shows a specific panel of cells/markers-based biosensor cellular profiling method to determine the compounds to be PI3K inhibitors. (A) The DMR signal of the known negative control LY303511 of the PI3K inhibitor LY294002 in the quiescent A431 cells; (B) The DMR signal of the known negative control LY303511 of the PI3K inhibitor LY294002 in lung cancer cell line A549; (C) The DMR modulation index of the known negative control LY303511 of the PI3K inhibitor LY294002 against panels of markers across the two cell lines (pinacidil, forskolin, and histamine for A549 cells, and EGF for A431 cells).

FIG. 13A-13C shows a specific panel of cells/markers-based biosensor cellular profiling method to determine the compounds to be PI3K inhibitors. (A) The DMR modulation index of the known weak PI3K inhibitor resveratrol against panels of markers across the two cell lines (pinacidil, forskolin, and histamine for A549 cells, and EGF for A431 cells). (B) The DMR modulation index of the known DNA-PK inhibitor III 1-(2-Hydroxy-4-morpholin-4-yl-phenyl)ethanone against panels of markers across the two cell lines (pinacidil, forskolin, and histamine for A549 cells, and EGF for A431 cells). (C) The DMR modulation index of the known DNA-PK inhibitor II 2-(Morpholin-4-yl)-benzo[h]chromen-4-one against panels of markers across the two cell lines (pinacidil, forskolin, and histamine for A549 cells, and EGF for A431 cells).

FIG. 14A-14B shows a specific panel of cells/markers-based biosensor cellular profiling method to determine the compounds to be PI3K inhibitors. (A) The DMR modulation index of the known ROCK inhibitor Y27632 against panels of markers across the two cell lines (pinacidil, forskolin, and histamine for A549 cells, and EGF for A431 cells). (B) The DMR modulation index of the known ROCK and PKA dual inhibitor H-89 against panels of markers across the two cell lines (pinacidil, forskolin, and histamine for A549 cells, and EGF for A431 cells).

FIG. 15A-15B shows a high resolution panel of cells/markers-based biosensor cellular profiling method to separate ROCK inhibitors from the PI3K inhibitors. (A) The DMR modulation index of the known PI3K inhibitor LY294002 against panels of markers across the four cell lines (pinacidil, poly(I:C), PMA, SLIGKV-amide, forskolin, and histamine for A549 cells; epinephrine (Epi), nicotinic acid (NA), EGF and histamine (His) for A431 cells; EGF, IGF-1, mallotoxin (MTX) and neurotensin (NT) for HT-29 cells; and ODN2006 for HepG2 cells). (B) The DMR modulation index of the known ROCK inhibitor Y-27632 against the same panels of markers across the four cell lines. Both kinase inhibitors were examined at 10 micromolar.

FIG. 16 shows a flow chart showing methods to screen Rho kinase inhibitors.

FIG. 17 shows a flow chart showing methods to screen Rho kinase inhibitors.

FIG. 18 shows a flow chart showing methods to identify phenotypic and poly-pharmacology of a Rho kinase inhibitor.

FIG. 19 The optical biosensor DMR signals of human lung cancer cell line A549 in response to a well-known Rho kinase inhibitor Y27631 (10 micromolar) in the absence (“buffer”) and presence of two compounds (the Rho kinase inhibitor Y27632, and another Rho kinase inhibitor “compound”). Either of these two inhibitors at 10 micromolar was used to pretreat the A549 cells for ˜1 hr, before the stimulation with Y27632. The desensitization of cells to the second stimulation with Y27632 is an indicator that the compound used for the pretreatment of the cells is a Rho kinase inhibitor.

FIG. 20 shows the optical biosensor DMR signals of human lung cancer cell line A549 in response to a well-known ATP-sensitive potassium ion channel opener pinacidil (10 micromolar) in the absence (“buffer”) and presence of two compounds (the Rho kinase inhibitor Y27632, and another Rho kinase inhibitor “compound”). Either of these two inhibitors at 10 micromolar was used to pretreat the A549 cells for ˜1 hr, before the stimulation with pinacidil. The desensitization of cells to the subsequent stimulation with pinacidil is an indicator that the compound used for the pretreatment of the cells is a Rho kinase inhibitor. Pinacidil triggered an optical response containing contributions from the Rho kinase pathway; and thus the inhibition of ROCK activity by the compound impaired the pinacidil DMR signal.

FIG. 21A-21C shows a specific panel of cells/markers-based biosensor cellular profiling method to determine the compounds to be Rho kinase inhibitors. (A) The DMR signal of a Rho kinase inhibitor Y-27632 in the lung cancer cell line A549; (B) The DMR signal of Y-27632 in the liver cell line HepG2; (C) The DMR modulation index of Y-27632 against panels of markers across the two cell lines (pinacidil, poly(IC), PMA, SLIGKV-amide, forskolin, and histamine for A549 cells, and ODN2006 for HepG2 cells.

FIG. 22A-22C shows a specific panel of cells/markers-based biosensor cellular profiling method to determine the compounds to be Rho kinase inhibitors. (A) The DMR signal of a Rho kinase inhibitor III Rockout in the lung cancer cell line A549; (B) The DMR signal of the Rho kinase inhibitor III Rockout in the liver cell line HepG2; (C) The DMR modulation index of the Rho kinase inhibitor III Rockout against panels of markers across the two cell lines (pinacidil, poly(IC), PMA, SLIGKV-amide, forskolin, and histamine for A549 cells, and ODN2006 for HepG2 cells.

FIG. 23A-23C shows a specific panel of cells/markers-based biosensor cellular profiling method to determine the compounds to be Rho kinase inhibitors. (A) The DMR signal of a Rho kinase inhibitor IV in the lung cancer cell line A549; (B) The DMR signal of the Rho kinase inhibitor IV in the liver cell line HepG2; (C) The DMR modulation index of the Rho kinase inhibitor IV against panels of markers across the two cell lines (pinacidil, poly(IC), PMA, SLIGKV-amide, forskolin, and histamine for A549 cells, and ODN2006 for HepG2 cells.

FIG. 24A-24C shows a specific panel of cells/markers-based biosensor cellular profiling method to determine the compounds to be Rho kinase inhibitors. (A) The DMR signal of CDK1/2 inhibitor III in the lung cancer cell line A549; (B) The DMR signal of the CDK1/2 inhibitor III in the liver cell line HepG2; (C) The DMR modulation index of the CDK1/2 inhibitor III against panels of markers across the two cell lines (pinacidil, poly(IC), PMA, SLIGKV-amide, forskolin, and histamine for A549 cells, and ODN2006 for HepG2 cells.

### DETAILED DESCRIPTION

### A. PI3Ks

PI3Ks constitute a large group of dual lipid and protein kinases and include three primary classes: I, II and III, according to their differences in sequence homology, substrate preference and function. Each primary class of PI3K is involved in distinct cellular signaling processes and can be further divided according to either sequence homology or regulatory mechanism. Class I PI3Ks are heterodimers constituting a catalytic subunit and a regulatory subunit. The catalytic subunits of class I PI3K contain four isoforms: p110α, p110β, p110δ and p110γ, encoded by four distinct genes, termed Pik3ca, Pik3cb, Pik3cd and Pik3cg, respectively. Corresponding to the catalytic subunits, isoforms of Class I PI3K are denoted PI3Kα, PI3Kβ, PI3δ and PI3Kγ. Based on their connections with regulatory subunits and activation mechanism, the four isoforms of class I PI3K can be further grouped into two categories: class IA (PI3Kα, PI3Kβ, and PI3Kδ) and class IB (PI3Kγ). Class IA PI3K isoforms can be activated by receptor tyrosine kinases (RTKs). Associated regulatory subunits of Class IA PI3K include p85α, p85β, p55α, p55γ and p50α. The PI3K regulatory subunit maintains the p110 catalytic subunit in a low-activity state in quiescent cells and mediates its activation by direct interaction with phosphotyrosine residues of activated growth factor receptors or adaptor proteins. Direct binding of p110 to the activated Ras protein (also induced by growth factor stimulation) further stimulates PI3K activity. The unique catalytic subunit of class IB PI3K is p110γ which is exclusively activated by G-proteins coupled receptor (GPCRs) and can specifically bind to adaptors unrelated to p85 protein. Pathophysiologic studies have unfolded the close connections between PI3Kα with oncogenesis, PI3Kβ with thrombosis, PI3Kδ with immune function and PI3Kγ with inflammation. Class II PI3Ks are monomers, lack of regulatory subunits, including three isoforms in human: PI3K-C2α, PI3K-C2β and PI3K-C2γ, the first two are expressed in most issues while the last one is restrictedly expressed in liver. Class III PI3Ks are heterodimers, consisting of a regulatory subunit (p150) and a catalytic subunit (Vps34). Vps34 shares significant sequence similarity with the catalytic subunit of class I PI3Ks. The class II and III PI3-Ks play a key role in intracellular trafficking through the synthesis of PI(3)P and PI(3,4)P2.

PI3K is activated by receptor tyrosine kinases and other cell-surface receptors to synthesize the lipid second messenger PIP3 (phosphatidylinositol-3,4,5-trisphosphate), although different classes of PI3Ks have preferences on phospholipids as substrates. PIP3, in turn, acts as a docking site at the plasma membrane that recruits and activates proteins containing phospholipid-binding domains. These downstream PI3K effectors include (i) protein kinases that promote cell growth, survival and proliferation, such as Akt (also known as protein kinase B), PDK1 (phosphoinositide-dependent kinase 1) and the Tec family kinases; (ii) GAPs (GTPase-activating proteins) and GEFs (guanine nucleotide-exchange factors) that regulate GTPases mediating cell motility and membrane trafficking; and (iii) scaffolding proteins that nucleate the assembly of key signaling complexes. PKB/Akt is a central node in cell signaling downstream and regulates a wide range of proteins, such as glycogen synthase kinase-3 (GSK3), Bcl-2-associated death promoter (BAD), caspase 9, p70 S6-kinase (p70S6k), eIF4E-binding proteins (4E-BP1) and members of the Forkhead box transcription factors (FOXO), either in positive or negative manners. In addition, the activation of the PI3K pathway is negatively regulated by two phosphoinositide phosphatases, PTEN and SHIP. PTEN (phosphatase and tensin homolog) is a 3′ phosphatase that dephosphorylates the 3′OH group of PIP3 and converts it to PIP2, thus acts as a tumor suppressor. The PI3K pathway, bifurcating at many points and integrating signals from many other pathways like those that involve Ras and p53, is a key transduction cascade of the cellular signaling network. In this way, PI3K utilizes a single second messenger, PIP3, to link extracellular hormonal cues to intracellular signaling proteins that control diverse cellular processes.

The PI3K isoforms not only give rise to distinct substrate specificities, but also expression patterns and modes of regulation. The expression patterns and functions of PI3K isoforms are quite distinct in different types of cells. Constitutive activation of PI3K and loss of function of PTEN often coexist in various cancers. Emerging evidence suggests that the PI3K pathway is among the most commonly activated signaling pathways in cancer. First, the p110α isoform of PI3K is activated by mutation at high frequency in a range of primary tumors, and it is estimated to be ˜15% at a cumulative frequency across all cancer types surveyed to date, suggesting that p110α is likely to be the most commonly mutated kinase in the human genome. Secondly, the phosphatase PTEN, which antagonizes PI3K signaling by dephosphorylating PIP3, is a well-characterized tumor suppressor that is frequently inactivated by mutation, gene deletion or epigenetic silencing. In addition, PI3K is allosterically activated by the oncogene Ras, and many tyrosine kinases that activate PI3K are themselves the target of mutations or amplification in cancer. Together, these observations reveal a nexus of genetic alterations in cancer that each stimulate PI3K signaling, suggesting that PI3K activation is likely to be an essential step in tumorigenesis. The non-α isoforms of p110 do not show cancer specific mutations. However, they are often differentially expressed in cancer and, in contrast to p110α, wild-type non-α isoforms of p110 are oncogenic when overexpressed in cell culture. Disclosed herein are methods to characterize cells, particularly cells having deregulated PI3K pathway activity.

Cancers with mutations that activate kinases are most likely to be effectively treated with appropriate inhibitors of these particular kinases. The strategy of targeting kinases in certain crucial signaling pathways to develop drugs for the treatment of human diseases is greatly supported by successful examples of kinase inhibitors, including Imatinib, Gefitinib, Erlotinib, Sorafenib, Dasatinib, Sunitinib, and Lapatinib. The isoforms of p110 have become promising drug targets. Efforts are underway to develop small molecule PI3K inhibitors for the treatment of inflammation and autoimmune disease (p110δ, p110γ, and mTOR), thrombosis (p110β), viral infection (the PIKKs), and cancer (p110α, mTOR, and others).

A key challenge in targeting the PI3-K family with drugs is to understand how individual PI3-K isoforms control normal physiology, as this defines the therapeutic window for targeting a specific isoform. Genetic approaches to uncouple the action of PI3-K isoforms have been frustrated by the complex coordinate regulation of these enzymes. Homozygous deletion of either p110α or p110β (the two most widely expressed PI3-Ks) leads to embryonic lethality in mice. Heterozygous deletion of these isoforms is complicated by a compensatory down regulation of the p85 regulatory subunit. Knockout of p85 isoforms induces a paradoxical increase in PI3-K signaling, reflecting the fact that p85 both promotes PI3-K activity (by stabilizing the p110 catalytic subunit) and inhibits it (by reducing basal activity and sequestering essential signaling complexes). A similar effect has been observed among the PIKKs, where a deficiency in DNA-PK alters the expression of ATM and hSmg-1. In addition to these compensatory mechanisms, PI3-Ks possess kinase-independent signaling activities that can cause inhibitors and knockouts to induce different phenotypes. For example, p110γ knockout mice develop cardiac damage in response to chronic pressure overload, whereas mice bearing a p110γ kinase-dead allele do not. In this case, the difference was traced to an allosteric activation of PDE3B by p110γ that is disrupted in the knockout but unaffected by the kinase-dead allele or an inhibitor.

Cell-permeable small molecule inhibitors make it possible to directly assess the phenotypic consequences of inhibiting a kinase with a drug in a physiologically relevant model system. The challenge for pharmacological target validation is that few well-characterized, selective kinase inhibitors are known. This has been particularly true for the PI3-Ks, as the two primary pharmacological tools available, wortmannin and LY294002, are broadly active within the family.

PI3K kinase inhibitors are mostly discovered using a variety of in vitro biochemical inhibition assays. Methods for determining PI3K substrate phosphorylation levels in live cells are also developed for measuring the cellular activity of PI3Ks, and for screening PI3K inhibitors. However, since many potential substrates of PI3K are also potentially phosphorylated by several other protein kinases, the degree of phosphorylation observed in cell extracts is not necessarily an accurate reflection of the activity of the PI3K. Therefore, establishing the level of PI3K activity in cells, including the effects of potential inhibitors, has been problematic.

Disclosed herein are methods related to label-free biosensor cellular assays. A label-free biosensor cellular assays involve—a biological component (i.e., live cells), a detector element, and a transducer associated with both components. Depending on the types of transducers used, label-free biosensors for whole cell sensing can be largely classified into three categories: acoustic, electrical and optical biosensors.

Disclosed herein are methods to screen PI3K inhibitors using label-free biosensor cellular assays, particularly to identify PI3K subtype selectivity of PI3K inhibitors using the biosensor cellular profiling assays.

### B. Rho GTPases and Rho Kinases

Rho GTPases represent a subset of the larger Ras superfamily and consist of over 20 intracellular signaling proteins. They are often referred to as the small GTPases because of their ˜20 kD size, with the most thoroughly characterized members being RhoA, Cdc42, and Rac1. They are ubiquitously expressed proteins known to be molecular switches for the transduction of signals from external stimuli through the activation of integrins, growth factor receptors, ion channels, and G-protein coupled receptors. The activation of the GTPase by receptor or non-receptor dependent mechanisms mediates the transition between an active GTP bound state and an inactive GDP bound state. Numerous studies have demonstrated the important roles of Rho GTPases in the regulation of gene transcription, cell proliferation, migration, cell division, and cell shape change. Thus, identification of Rho kinase inhibitor could lead to new drugs. Disclosed herein are methods to screen for Rho kinase inhibitors.

The regulation of Rho GTPase activity is controlled by a group of regulatory proteins that are specific for each family member. These regulatory proteins fall into three general categories: Guanine nucleotide exchange factors (GEFs), GTPase activating proteins (GAPs), and guanine nucleotide dissociation inhibitors (GDIs). GEFs act to enhance the exchange of GDP for GTP and thus activate GTPases. GAPs control the hydrolysis of GTP to GDP and are integral in maintaining an appropriate basal level of activity. GDI proteins have varying affinities for different GTPases and keep them from being activated. The differential regulation by the GEFs, GAPs and GDIs allows for selective spatial activation/inactivation of Rho proteins in the cells.

The Rho proteins have over 60 known downstream effectors, which determine the outcome of the activation for a given Rho GTPase protein. The downstream target of the Rho GTPases dictates if the activation plays a role in cell morphology, polarity, vesicular trafficking, or cell cycle control. Activated RhoA influences the tension of cell attachment to the substrate, which is supported by the actin abundant stress fiber in the cell. Rac1 and Cdc 42, on the other hand, affect the formation of peripheral projections rich in actin like lamellipodia and filapodia. The most common action of Rho GTPase activation on mammalian cells is through the reorganization of actin cytoskeleton. A number of Rho downstream targets have been identified, including PKN which is protein kinase C (PKC)-like kinase, Rhotekin, Citron, PIPS-kinase (phosphatidylinositol 4-phosphate 5-kinase), and p140mDia, which regulate actin polymerization.

Rho kinase, also referred to as ROCK, is the major effecter of RhoA. ROCK is a serine/threonine protein kinase of ˜160 kDa which corresponds to gene products, ROCK I and ROCK II (rho-associated coiled-coil containing kinase-1 and -2, also known as Rokb/p160ROCK and Roka, respectively). The two kinases have 64% overall identity in humans with 89% identity in the catalytic kinase domain. Both kinases contain a coiled-coil region and a pleckstrin homology (PH) domain split by a C1 conserved region. The two ROCKs have spatially differential expressions. Rho kinase is autoregulated by its COOH-terminal domain, which folds back onto the active site to inhibit its kinase activity. Only the active GTP bound form of RhoA binds to ROCK and blocks the inactivation of the protein. As long as the active form of Rho is bound to ROCK, the kinase remains active. Rho kinase can also be activated by arachidonic acid and sphingosylphosphorylcholine. The cleavage of the inhibitory COOH-terminus by caspases can result in an increase in ROCK activity during apoptosis.

The serine/threonine kinases ROCK1 and ROCK2 are direct targets of activated Rho GTPases, and aberrant rho/ROCK signaling has been implicated in a number of human diseases. ROCK1 and ROCK2 are closely related members of the AGC subfamily of enzymes that are activated downstream of activated Rho in response to a number of extracellular stimuli, including growth factors, integrin activation, and cellular stress. ROCK activation leads to a concerted series of events that promote force generation and morphological changes. These events contribute directly to a number of actin-myosin-mediated processes, such as cell motility, adhesion, smooth muscle contraction, neurite retraction, and phagocytosis. In addition, ROCK kinases play roles in proliferation, differentiation, apoptosis, and oncogenic transformation, although these responses can be cell type-dependent.

The activation of ROCK results in the subsequent phosphorylation of a number of different downstream targets. The most well known target of Rho kinase is myosin light chain (MLC). Myosin phosphatase is also phosphorylated by Rho kinase and this interaction causes an increase in phosphorylated MLC. In addition, ROCK phosphorylates LIM kinase-1 and kinase-2 (LIMK1 and LIMK2) at conserved threonines in their activation loops, increasing LIMK activity, and the subsequent phosphorylation of cofilin proteins, which blocks their F actin-severing activity. Many other proteins involved in actin cytoskeleton rearrangement are phosphorylated by ROCK.

The ROCK enzymes play key roles in multiple cellular processes, including cell morphology, stress fiber formation and function, cell adhesion, cell migration and invasion, epithelial-mesenchymal transition, transformation, phagocytosis, apoptosis, neurite retraction, cytokinesis, and cellular differentiation. As such, ROCK kinases represent potential targets for the development of inhibitors to treat a variety of disorders, including cancer, hypertension, vasospasm, asthma, preterm labor, erectile dysfunction, glaucoma, atherosclerosis, myocardial hypertrophy, and neurological diseases.

Asthma is a chronic inflammatory airways disease characterized by early and late asthmatic reactions that are associated with infiltration and activation of inflammatory cells in the airways and airway hyperresponsiveness to a variety of stimuli, including neurotransmitters and inflammatory mediators. In asthma, inflammatory mediators that are released in the airways by recruited inflammatory cells and by resident structural cells result in airway hyperresponsiveness caused by increased bronchoconstriction. In addition, chronic inflammation appears to drive remodelling of the airways that contributes to the development of fixed airway obstruction and airway hyperresponsiveness in chronic asthma. Airway remodelling includes several key features such as excessive deposition of extracellular matrix proteins in the airway wall (fibrosis) and increased abundance of contractile airway smooth muscle encircling the airways. Airway hyperresponsiveness could be explained, in part, by increased contraction of airway smooth muscle, caused either by an intrinsic functional change in the muscle or by alterations in the neurogenic and non-neurogenic control of muscle function. In addition, development of airway hyperresponsiveness is underpinned by physical changes in the airways, such as damage of the epithelial layer, mucosal swelling, goblet cell hyperplasia and remodeling of the airway wall. Airway remodeling is typically characterized by thickening of the lamina reticularis, augmented subepithelial extracellular matrix deposition (fibrosis), and increased abundance of contractile airway smooth muscle encircling the airways. Current asthma therapy fails to inhibit these features satisfactorily. Currently, treatment of acute and chronic features of allergic asthma is achieved primarily by β2-adrenoceptor agonists and corticosteroids. Acute bronchospasm, resulting from excessive airway smooth muscle contraction, can be satisfactorily reversed in most patients by inhaled β2-adrenoceptor agonists as they cause airway smooth muscle relaxation. Unfortunately, however, patients can develop tolerance to β2-adrenoceptor agonists, and these agents have minimal effects on airway inflammation and airway remodeling in vivo, despite reports that they can inhibit individual features of airway remodeling (e.g. airway smooth muscle proliferation) in vitro. Inhaled corticosteroids represent the mainstay for the control of several allergic diseases, including persistent mild, moderate and severe asthma, and are well known for their broad-spectrum of activities that reduce the intensity of inflammatory processes that characterize asthma. Unfortunately, however, several features of airway inflammation (e.g. neutrophilia) can be relatively insensitive to corticosteroid treatment. Moreover, corticosteroids are only partially effective in inhibiting features of airway remodeling. Thus, although corticosteroids effectively prevent several features of airway remodeling (fibrosis, airway smooth muscle thickening, mucus gland hypertrophy) they are poorly effective in reversing airway wall remodeling. The limitations associated with β2-adrenoceptor agonist and corticosteroid treatment have urged to the investigation and identification of alternative drug targets. Amongst those, Rho kinase has emerged as a potential target for the treatment of airway hyperresponsiveness in asthma. Rho-kinase is an effector molecule of RhoA, a monomeric GTP-binding protein, and causes Ca2+ sensitization via inactivation of myosin phosphatase. The major physiological functions of Rho-kinase include contraction, migration, and proliferation in cells. These actions are thought to be related to the pathophysiological features of asthma, i.e., airflow limitation, airway hyperresponsiveness, β-adrenergic desensitization, eosinophil recruitment and airway remodeling.

### C. Methods

The methods disclosed herein, as well as the compositions and compounds which can be used in the methods, can arise from a number of different classes, such as materials, substance, molecules, and ligands. Also disclosed is a specific subset of these classes, unique to label free biosensor assays, called markers, for example, EGF as a marker for EGFR activation.

It is understood that mixtures of these classes, such as a molecule mixture are also disclosed and can be used in the disclosed methods.

In certain methods, unknown molecules, test molecules, drug candidate molecules as well as known molecules can be used.

In certain methods or situations, modulating or modulators play a role. Likewise, known modulators can be used.

In certain methods, as well as compositions, cells are involved, and cells can undergo culturing and cell cultures can be used as discussed herein.

The methods disclosed herein involve assays that use biosensors. In certain assays, they are performed in either an agonism or antagonism mode. Often the assays involve treating cells with one or more classes, such as a material, a substance, or a molecule. It is also understood that subjects can be treated as well, as discuss herein.

In certain methods, contacting between a molecule, for example, and a cell can take place. In the disclosed methods, responses, such as cellular response, which can manifest as a biosensor response, such as a DMR response, can be detected. These and other responses can be assayed. In certain methods the signals from a biosensor can be robust biosensor signals or robust DMR signals.

The disclosed methods utilizing label free biosensors can produce profiles, such as primary profiles, secondary profiles, and modulation profiles. These profiles and others can be used for making determinations about molecules, for example, and can be used with any of the classes discussed herein.

Also disclosed are libraries and panels of compounds or compositions, such as molecules, cells, materials, or substances disclosed herein. Also disclosed are specific panels, such as marker panels and cell panels.

The disclosed methods can utilize a variety of aspects, such as biosensor signals, DMR signals, normalizing, controls, positive controls, modulation comparisons, Indexes, Biosensor Indexes, DMR indexes, Molecule biosensor indexes, molecule DMR indexes, molecule indexes, modulator biosensor indexes, modulator DMR indexes, molecule modulation indexes, known modulator biosensor indexes, known modulator DMR indexes, marker biosensor indexes, marker DMR indexes, modulating the biosensor signal of a marker, modulating the DMR signal, potentiating, and similarity of indexes.

Any of the compositions, compounds, or anything else disclosed herein can be characterized in any way disclosed herein.

Disclosed are methods that rely on characterizations, such as higher and inhibit and like words.

In certain methods, receptors or cellular targets are used. Certain methods can provide information about signaling pathway(s) as well as molecule-treated cells and other cellular processes.

In certain embodiments, a certain potency or efficacy becomes a characteristic, and the direct action (of a drug candidate molecule, for example) can be assayed.

1. Methods to Screen PI3K Inhibitors

Disclosed herein are methods to screen and characterize phosphoinositide 3-kinase PI3K inhibitors using label-free biosensor cellular assays. The disclosed methods include three distinct methods and their combinations to screen PI3K inhibitors.

The three methods are: (1) molecules are screened with a label-free biosensor cellular assays against a PI3K inhibitor in a PI3K pathway-amplified cell, wherein the PI3K pathway-amplified cell is also a PI3K pathway deregulated cell or a PI3K pathway hyperactivated cell; (2) molecules are screened with a label-free biosensor cellular assays against a marker whose biosensor cellular response contains PI3K-associated pathway contributions in a PI3K pathway-amplified cell; and (3) molecules are assayed with a label-free biosensor cellular assays against panels of markers, each panel of markers for a cell type, whose biosensor index, particularly biosensor DMR index, is used as an indicator for the compounds being PI3K inhibitors. Particularly, in certain embodiments the disclosed methods can separate ROCK inhibitors from PI3k inhibitors. The disclosed methods are also to characterize cells regarding whether the PI3K pathway is deregulated or not.

The ability to examine the action and its cellular consequence of novel compounds within the context of an intact cell via their target proteins is crucial to determine the utility of such compounds for the treatment of disorders involving intracellular target proteins such as PI3K isoforms. Ideally the method used to establish cell-based activity should be specific for the target protein of interest, amenable to testing a large number of potential inhibitors, and quantitative to allow identification of preferred compounds.

Although the dominant paradigm in drug discovery is the concept of designing maximally selective ligands to act on individual drug targets, many effective drugs, particularly kinase inhibitors including PI3K inhibitors, act via modulation of multiple proteins rather than single targets (i.e., polypharmacology). In addition, drug pharmacology often displays cell- or tissue-dependency (i.e., phenotypic pharmacology). Although the cellular factors responsible for generating phenotypic profiles are largely unknown, there is substantial evidence that factors, including post-transcriptional modifications, compartmentalization of signaling cascades, receptor oligomerization and constitutive receptor activity, can influence drug pharmacology, leading to so-called “phenotypic pharmacology”. Thus, the primary modes of action of a drug or drug candidate molecule, including PI3K inhibitors, are a sum of at least several types of pharmacology—polypharmacology, phenotypic pharmacology and systems (integrative) pharmacology.

Label-free biosensor cellular assays often provide an integrated readout in a pathway-unbiased but pathway-sensitive manner. As a result, label-free biosensor cellular assays often reflect the complexity of receptor biology and drug pharmacology. Coupled with the non-specific nature of label-free biosensor as well as the complexity of cell biology (e.g., redundant signaling elements, and compensated feedback loops), a single target-based label-free biosensor cellular assay typically leads to high percentage false positives.

The disclosed methods combine three types of label-free biosensor cellular assays for screening potential PI3K inhibitors with few false positives. Since ROCK is often linked to the PI3K pathway and can be either upstream or downstream of PI3Ks in cell type-dependent and receptor-dependent manner, the disclosed methods can enable separation of ROCK inhibitors from PI3K inhibitors, and also leads to identification of PI3K inhibitors whose phenotypic pharmacology (i.e., primary modes of action in intact cells) is directly associated with their inhibitory activity on PI3Ks. The disclosed methods can also enable identification of cells whose PI3K pathway is amplified or not.

Also disclosed are any combinations of these methods. These combinations can be used to characterize a PI3K inhibitor in terms of their selectivity, specificity, as well as phenotypic pharmacology to be a PI3K inhibitor.

Also disclosed are methods to characterize the cell lines and determine whether the cell lines have PI3K pathway amplification or not. Specifically, the biosensor modulation index, or biosensor index of several well-characterized PI3K inhibitors can be generated against a panel of markers in a specific cell line. The sensitivity of the PI3K inhibitor biosensor index or modulation index to PI3K inhibitors, particularly, the modulation profiles for both receptor tyrosine kinase and GPCR marker-induced biosensor signals, can be used as a readout whether the cell line has a PI3K pathway amplification or deregulation or not.

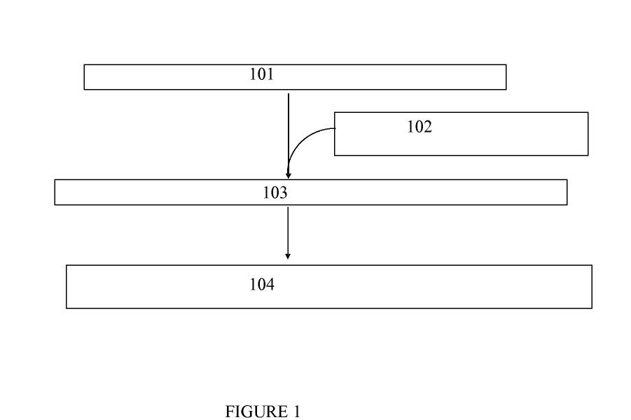

Disclosed herein are methods, as shown in FIG. 1, wherein molecules are screened with a label-free biosensor cellular assays against a PI3K inhibitor in a PI3K pathway amplified cell, wherein the desensitization of molecule-pretreated cells to the succeeding stimulation with the known PI3K inhibitor at a specific concentration (e.g., EC50, EC80, or EC100, or EC200) is used an indicator for the molecule to be a PI3K inhibitor. The molecule or compound can be introduced into the cultured cells at a specific time (e.g., 4 hr after attachment, 20 hr after attachment, 1 hr before the simulation with a PI3K inhibitor). This approach can be used for high throughput screening of PI3K inhibitors or PI3K pathway modulators. However, such an approach could lead to high false positives for PI3K inhibitors, due to the integrated nature and contributions of downstream signaling events to the PI3K inhibitor induced biosensor signal. The PI3K pathway amplified cells are the cells that have genetic mutations or gene expression alterations in the PI3K pathway cascades, including constitutively active K-Ras mutations, overexpression of growth factor receptors such as HER2, loss-of-function of PI3K antagonist protein such as PTEN, activating mutations in the PIK3CA gene, and overexpression of PI3K isoforms, that lead to the constitutive activity of PI3K pathway. Examples of such cells include, but not limited to, lung cancer cell lines A549 and H820 (cells having activating mutants of K-Ras), A4 and A7 cells (cells having low expression of PTEN), human giant-cell lung cancer cells 95C and 95D cells (cells having no PTEN proteins), and certain breast cancer cells including MDA-468 (cells having loss of PTEN), BT474 and SKBR3 (cells having amplification of HER2), MCF-7 and T47D (cells having mutated p110α), and others. The PI3K inhibitors are chosen from well-known and established PI3K inhibitors including, but not limited to, LY294002, and wortmannin.

Disclosed herein are methods, as seen in FIG. 2, wherein molecules are screened with a label-free biosensor cellular assay against a marker whose biosensor cellular response contains PI3K pathway contributions in a PI3K pathway-amplified cell, wherein the marker(s) are selected if their respective biosensor signals contain contributions from PI3K pathway. The similarity between the modulation profiles of a molecule and a well-known PI3K inhibitor is used as an indicator for the molecule to be a PI3K inhibitor. Such an approach is also amenable to high throughput screening. However, such an approach could also suffer high false positives for specific PI3K inhibitors. A variation of such approach can also be developed to reduce the false positives of screening for PI3K inhibitors. To do so, a panel of markers, each triggering biosensor signals containing contributions from PI3K pathways, can be used to generate the modulation indexes of both a well-known PI3K inhibitor and a compound. The similarity of the biosensor modulation index can be used as a readout for the compound to be PI3K inhibitor or not.

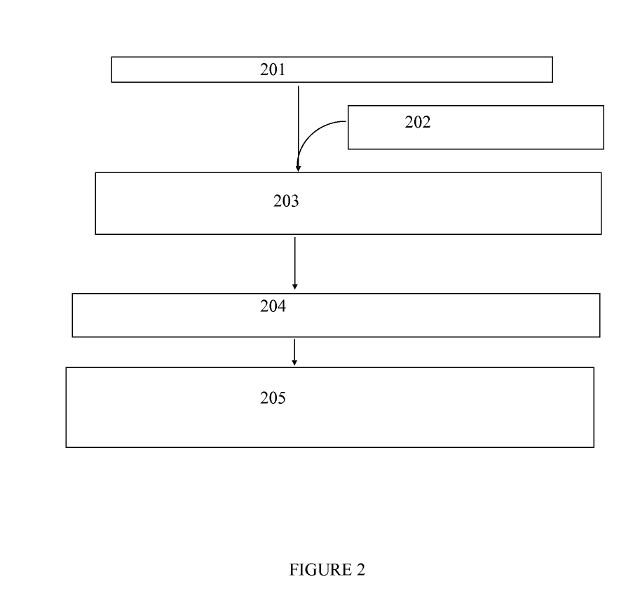

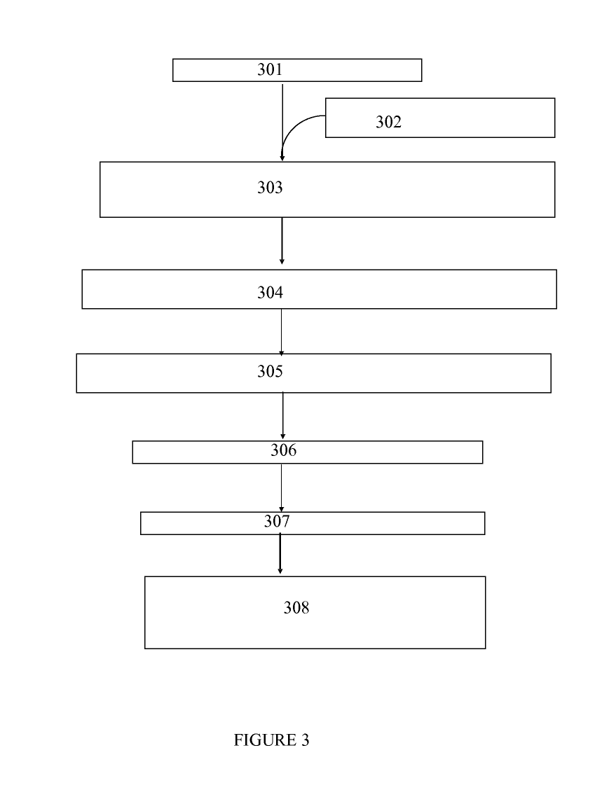

As shown in FIG. 3, in another disclosed method, molecules are assayed with label-free biosensor cellular assays against panels of markers, each panel of markers for a cell type, whose biosensor index, particularly biosensor DMR index, is used as an indicator for the compounds being PI3K inhibitors. Such an approach is well-suited to determine the polypharmacology and phenotypic pharmacology of a PI3K inhibitor. This approach will lead to low false positives for PI3K inhibitor screening, and also enable separation of ROCK inhibitors from PI3K inhibitors.

2. Methods to Screen ROCK Inhibitors

Disclosed herein are methods to screen Rho kinase(ROCK) inhibitors using label-free biosensor cellular assays. The disclosed methods include three distinct methods and their combinations to screen ROCK inhibitors—(1) molecules are screened with a label-free biosensor cellular assays against a Rho kinase inhibitor in a Rho kinase inhibitor responsive cell; (2) molecules are screened with a label-free biosensor cellular assays against a marker whose biosensor cellular response contains Rho kinase-associated pathway contributions in a Rho kinase inhibitor responsive cell; and (3) molecules are assayed with a label-free biosensor cellular assays against panels of markers, each panel of markers for a cell type, whose biosensor index, particularly biosensor DMR index, is used as an indicator for the compounds being Rho kinase inhibitors.

Rho kinase inhibitors are mostly discovered using a variety of in vitro biochemical inhibition assays. Methods for determining ROCK substrate phosphorylation levels in live cells are also developed for measuring the cellular activity of Rho kinase, and for screening Rho kinase inhibitors. However, since many potential substrates of ROCK are also potentially phosphorylated by several other protein Ser/Thr kinases, the degree of phosphorylation observed in cell extracts is not necessarily an accurate reflection of the activity of the ROCK enzymes. For example, the regulatory light chain component of myosin (MLC) can be phosphorylated at Ser19 by ROCK, but under certain conditions is also a substrate for additional kinases, including myosin light chain kinases (MLCK), myotonic dystrophy-related and cdc42-activated kinases (MRCK), and ZIPK. Therefore, establishing the level of ROCK activity in cells, including the effects of potential inhibitors, has been problematic.

Disclosed methods are related to label-free biosensor cellular assays. Disclosed methods are related to methods to screen and characterize molecules as ROCK inhibitors, as well as existing ROCK inhibitors using label-free biosensor cellular assays. Specifically, disclosed methods include three distinct methods and their combinations to screen molecules as Rho kinase inhibitors, as well as existing Rho kinase inhibitors.

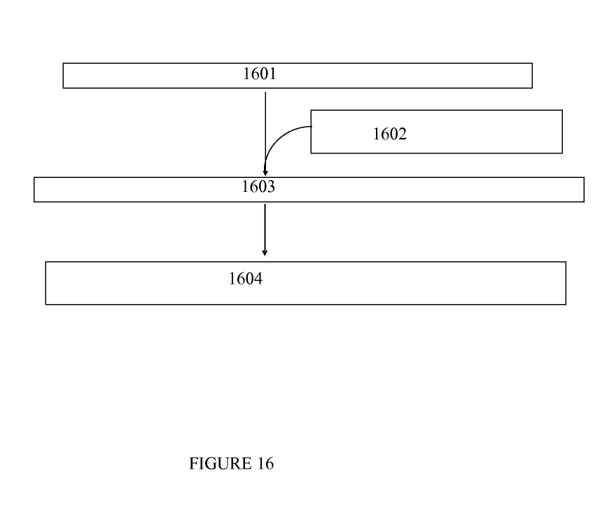

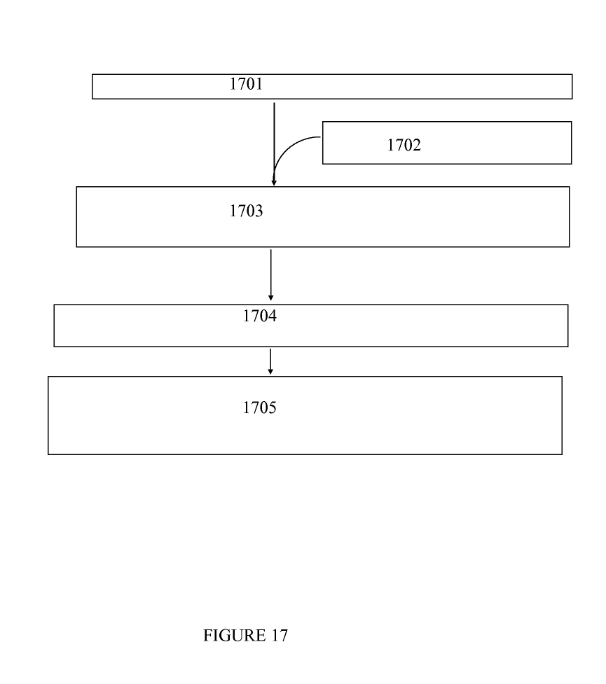

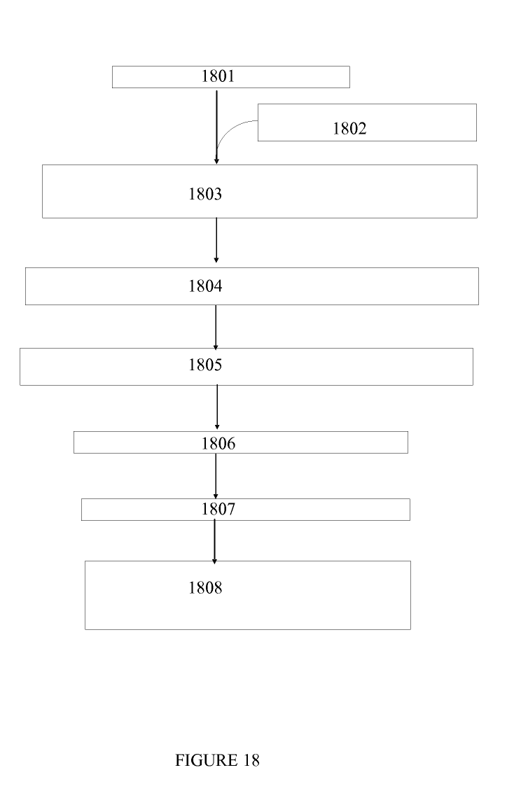

As shown in FIG. 16, in a disclosed method, molecules are screened with a label-free biosensor cellular assays against a Rho kinase inhibitor in a Rho kinase inhibitor responsive cell, wherein the desensitization of molecule-pretreated cells to the succeeding stimulation with the Rho kinase inhibitor at a specific concentration (e.g., EC50, EC80, or EC100, or EC200) is used an indicator for the molecule to be a ROCK inhibitor. The molecule or compound can be introduced into the cultured cells at a specific time (e.g., 4 hr after attachment, 20 hr after attachment, 1 hr before the simulation with a Rho kinase inhibitor). This approach can be used for high throughput screening of ROCK inhibitors or ROCK pathway modulators. However, such approach could lead to high false positives for specific ROCK inhibitors, due to the integrated nature and contributions of downstream signaling events to the ROCK inhibitor induced biosensor signal. The Rho kinase inhibitor responsive cells are the cells that give rise to a detectable biosensor output signal upon stimulation with a known Rho kinase inhibitor such as Y-27632 and H-89. Examples are A431, HT29 and A549 cells.

As shown in FIG. 2, in another disclosed method, molecules are screened with a label-free biosensor cellular assay against a marker whose biosensor cellular response contains Rho kinase-associated pathway contributions in a Rho kinase inhibitor responsive cell, wherein the marker(s) are selected if their respective biosensor signals contain contributions from ROCK pathways. The similarity between the modulation profiles of a molecule and a well-known ROCK inhibitor is used as an indicator for the molecule to be a ROCK inhibitor. Such approach is also amenable to high throughput screening. However, such approach could also suffer high false positives for specific ROCK inhibitors.

As shown in FIG. 3, in again another disclosed method, molecules are assayed with label-free biosensor cellular assays against panels of markers, each panel of markers for a cell type, whose biosensor index, particularly biosensor DMR index, is used as an indicator for the compounds being Rho kinase inhibitors. Such approach is well-suited to determine the polypharmacology and phenotypic pharmacology of a ROCK inhibitor.

Also disclosed are any combinations of these methods. These combinations can be used to certain a ROCK inhibitor in terms of their selectivity, specificity, as well as phenotypic pharmacology to be a ROCK inhibitor.

3. Specific Embodiments

Disclosed are methods of assaying a molecule comprising the steps: a. culturing a deregulated PI3K pathway cell line on a surface, wherein the surface can be used in a label free biosensor analysis, b. incubating the cell line with the molecule producing a molecule treated cell line, c. analyzing the molecule treated cell line with a label free biosensor producing a molecule data output, d. comparing the molecule data output to a known PI3K inhibitor data output in the same cell line, producing a molecule-PI3K inhibitor comparison.

Also disclosed are methods, wherein the known PI3K inhibitor data output was produced by incubating the PI3K inhibitor with the cultured deregulated PI3K cell line and analyzing the incubated cell line with a label free biosensor producing a PI3K inhibitor data output, and/or alone or in any combination with any limitation disclosed herein.

Also disclosed are methods, further comprising identifying a potential PI3K inhibitor when the comparison indicates that the molecule data output and the PI3K inhibitor data output are similar, and/or alone or in any combination with any limitation disclosed herein.

Disclosed are methods, wherein the deregulated PI3K pathway cell line is selected from a K-Ras activating mutant cell line, a HER2 overexpressed cell line, a PTEN loss-of-function cell line, or a PI3KCA activating mutant cell line, wherein the K-Ras activating mutant cell line is A549 cell line, or H820 cell line, wherein the PTEN loss-of-function cell line is A4 cell line, A7 cell line, human giant-cell lung cancer cell line 95C, human giant-cell lung cancer cell line 95D, or breast cancer cell line MDA-468, wherein the HER2 overexpressed cell line is BT474, or SKBR3, wherein the PI3KC activating mutant cell line is MCF-7, or T47D, wherein the known PI3K inhibitor comprises LY294002, or wortmannin, and/or alone or in any combination with any limitation disclosed herein.

Disclosed are methods, further comprising incubating the molecule with a normal PI3K pathway cell line, analyzing the molecule-treated cell line with a label free biosensor producing a second molecule data output, and comparing the second molecule data output to a PI3K inhibitor data output produced in the same cell line, and/or alone or in any combination with any limitation disclosed herein.

Also disclosed are methods, wherein the cell line overexpresses EGF receptor, wherein the cell line is the A431 cell line, and/or alone or in any combination with any limitation disclosed herein.

Disclosed are methods of assaying a molecule comprising the steps: a. culturing a deregulated PI3K pathway cell line on a surface, wherein the surface can be used in a label free biosensor analysis, b. incubating the cell line with a marker and the molecule producing an incubating cell line, c. analyzing the incubating cell line with a label free biosensor producing a marker-molecule data output, d. comparing the marker-molecule data output to a marker-PI3K inhibitor data output in the same cell line, producing a marker-molecule/marker-PI3K inhibitor comparison.

Also disclosed are methods, wherein the marker-PI3K inhibitor data output was produced by incubating the marker and PI3K inhibitor with a cultured deregulated PI3K cell line and analyzing the incubated cell line with a label free biosensor producing a marker-PI3K inhibitor data output, and/or alone or in any combination with any limitation disclosed herein.

Also disclosed are methods, further comprising identifying a potential PI3K inhibitor when the comparison indicates that the marker-molecule data output and the marker-PI3K inhibitor data output are similar, further comprising incubating the marker and molecule with a normal PI3K pathway cell line, analyzing the marker-marker treated cell line with a label free biosensor producing a second marker-molecule data output, and comparing the second marker-molecule data output to a marker-PI3K inhibitor data output produced in the same cell line, further comprising incubating the marker and molecule with a Toll-like receptor cell line, analyzing the Toll-like receptor incubating cell line with a label free biosensor producing a third marker-molecule data output, and comparing the third marker-molecule data output to a marker-PI3K inhibitor data output produced in the same cell line, and/or alone or in any combination with any limitation disclosed herein.

Also disclosed are methods, wherein the Toll-like receptor cell line expresses TLR9, wherein the cell line is a HepG2 cell line, wherein the marker comprises a TLR9 agonist, wherein the marker comprises EGF, histamine, or forskolin, wherein the deregulated PI3K pathway cell line is selected from a K-Ras activating mutant cell line, a HER2 overexpressed cell line, a PTEN loss-of-function cell line, or a PI3KCA activating mutant cell line, wherein the K-Ras activating mutant cell line is A549 cell line, or H820 cell line, wherein the PTEN loss-of-function cell line is A4 cell line, A7 cell line, human giant-cell lung cancer cell line 95C, human giant-cell lung cancer cell line 95D, or breast cancer cell line MDA-468, wherein the HER2 overexpressed cell line is BT474, or SKBR3, wherein the PI3KC activating mutant cell line is MCF-7, or T47D, wherein the PI3K inhibitor comprises LY294002, quercetin, or wortmannin, and/or alone or in any combination with any limitation disclosed herein.

Also disclosed are methods, further comprising assaying the molecule for ROCK pathway activity, and/or alone or in any combination with any limitation disclosed herein.

Disclosed are methods, wherein the step of assaying comprises, a. culturing a ROCK inhibitor responsive cell line on a surface, wherein the surface can be used in a label free biosensor analysis, b. incubating the cell line with the molecule producing an incubating cell line, c. analyzing the incubating cell line with a label free biosensor producing a molecule data output, d. comparing the molecule data output to a known ROCK inhibitor data output in the same cell, producing a molecule-ROCK inhibitor comparison.

Disclosed are methods, wherein the ROCK inhibitor responsive cell line comprises a cell line in which inhibiting the basal activity of ROCKs by the known ROCK inhibitor produces a robust biosensor signal, wherein the cell line is selected from A431, A549, or HT29, wherein the method is performed independently with at least 2 markers, and/or alone or in any combination with any limitation disclosed herein.

Disclosed are methods, wherein the markers comprise 1) an activator of the TLR signaling pathway, 2) an activator of Gq-, Gi- and G12/13-mediated signaling, 3) an activator of Rho- or JAK-mediated signaling, 4) an activator of the PKC pathway, and 5) an activator of the cAMP-PKA or cAMP-EPAC-PI3K pathway, and/or alone or in any combination with any limitation disclosed herein.

Disclosed are methods, wherein the activator of the TLR signaling pathway comprises an agonist for Toll-like receptor(s), wherein the agonist for Toll-like receptor(s) comprises poly(IC), or ODN2006, wherein the activator of Gq-, Gi- and G12/13-mediated signaling comprises an agonist for protease activated receptor subtype 2 (PAR2), or an agonist for histamine H1 receptor, wherein the activator of Rho- or JAK-mediated signaling comprises an opener for endogenous ATP-sensitive potassium (KATP) ion channel, wherein the activator of the PKC pathway comprises an activator for protein kinase C (PKC), wherein the activator of the cAMP-PKA or cAMP-EPAC-PI3K pathway comprises an activator for adenylyl cyclases, wherein the method is performed independently with at least 2 markers, wherein the markers comprise an agonist for β2-adrenergic receptor, an agonist for the endogenous GPR109A, an EGFR agonist, and an agonist for histamine H1 receptor, wherein the markers are epinephrine for the β2-adrenergic receptor, nicotinic acid for the GPR109A, EGF for the EGFR, and histamine for the histamine H1 receptor, wherein the cell line comprises the HT29 cell line, wherein the method is performed independently with at least 2 markers, wherein the markers comprise an activator of the PKC and MAPK pathways, an activator of IGF1R mediated signaling, an activator of a hERG pathway, and an activator of Gq and Gi-mediated signaling and EGFR transactivation, wherein the activator of the PKC and MAPK pathways comprises the EGFR agonist EGF, the activator of IGF1R mediated signaling is the IGF1R agonist IGF1, the activator of a hERG pathway comprises the hERG activator mallotoxin, and the activator of Gq and Gi-mediated signaling and EGFR transactivation is the NTS1 agonist neurotensin, wherein the marker is added at a concentration of at least an EC70, an EC80, an EC 90, and EC95, and an EC99, wherein the ROCK inhibitor Y17631 and the PI3K inhibitor LY-294002, and/or alone or in any combination with any limitation disclosed herein.

Disclosed are methods of assaying a molecule comprising the steps: a. culturing four different cell lines on a surface, wherein the surface can be used in a label free biosensor analysis, wherein the four cell lines are A431, A549, HT29, and HepG2, b. incubating each cell line with the molecule producing four incubating cell lines, c. analyzing each incubating cell line with a label free biosensor producing a molecule data output for each incubating cell line, d. comparing the molecule data output to a ROCK inhibitor data output in the same cell line, producing a molecule-ROCK inhibitor comparison for each cell line.

Disclosed are methods, wherein the ROCK inhibitor data output was produced by incubating the ROCK inhibitor with each cell line and analyzing each incubated cell line with a label free biosensor producing a ROCK inhibitor data output for each cell line, wherein the Rho Kinase inhibitor comprises ROCK kinase inhibitor III Rockout, Rho kinase inhibitor IV, or Y-27632, and/or alone or in any combination with any limitation disclosed herein.

Also disclosed are methods, further comprising producing a PI3K inhibitor data output, and producing a molecule-PI3K kinase inhibitor comparison for each cell line, and/or alone or in any combination with any limitation disclosed herein.

### D. Acoustic Biosensors

Acoustic biosensors such as quartz crystal resonators utilize acoustic waves to characterize cellular responses. The acoustic waves are generally generated and received using piezoelectric. An acoustic biosensor is often designed to operate in a resonant type sensor configuration. In a typical setup, thin quartz discs are sandwiched between two gold electrodes. Application of an AC signal across electrodes leads to the excitation and oscillation of the crystal, which acts as a sensitive oscillator circuit. The output sensor signals are the resonance frequency and motional resistance. The resonance frequency is largely a linear function of total mass of adsorbed materials when the biosensor surface is rigid. Under liquid environments the acoustic sensor response is sensitive not only to the mass of bound molecules, but also to changes in viscoelastic properties and charge of the molecular complexes formed or live cells. By measuring the resonance frequency and the motion resistance of cells associated with the crystals, cellular processes including cell adhesion and cytotoxicity can be studied in real time.

### E. Electrical Biosensors

Electrical biosensors employ impedance to characterize cellular responses including cell adhesion. In a typical setup, live cells are brought in contact with a biosensor surface wherein an integrated electrode array is embedded. A small AC pulse at a constant voltage and high frequency is used to generate an electric field between the electrodes, which are impeded by the presence of cells. The electric pulses are generated onsite using the integrated electric circuit; and the electrical current through the circuit is followed with time. The resultant impedance is a measure of changes in the electrical conductivity of the cell layer. The cellular plasma membrane acts as an insulating agent forcing the current to flow between or beneath the cells, leading to quite robust changes in impedance. Impedance-based measurements have been applied to study a wide range of cellular events, including cell adhesion and spreading, cell micromotion, cell morphological changes, and cell death, and cell signaling.

### F. Optical Biosensors

Optical biosensors primarily employ a surface-bound electromagnetic wave to characterize cellular responses. The surface-bound waves can be achieved either on gold substrates using either light excited surface plasmons (surface plasmon resonance, SPR) or on dielectric substrate using diffraction grating coupled waveguide mode resonances (resonance waveguide grating, RWG). For SPR including mid-IR SPR, the readout is the resonance angle at which a minimal in intensity of reflected light occurs. Similarly, for RWG biosensor including photonic crystal biosensors, the readout is the resonance angle or wavelength at which a maximum incoupling efficiency is achieved. The resonance angle or wavelength is a function of the local refractive index at or near the sensor surface. Unlike SPR which is limited to a few of flow channels for assaying, RWG biosensors are amenable for high throughput screening (HTS) and cellular assays, due to recent advancements in instrumentation and assays. In a typical RWG, the cells are directly placed into a well of a microtiter plate in which a biosensor consisting of a material with high refractive index is embedded. Local changes in the refractive index lead to a dynamic mass redistribution (DMR) signal of live cells upon stimulation. These biosensors have been used to study diverse cellular processes including receptor biology, ligand pharmacology, and cell adhesion.

The present invention preferably uses resonant waveguide grating biosensors, such as Corning Epic® systems. Epic® system includes the commercially available wavelength integration system, or angular interrogation system or swept wavelength imaging system (Corning Inc., Corning, N.Y.). The commercial system consists of a temperature-control unit, an optical detection unit, with an on-board liquid handling unit with robotics, or an external liquid accessory system with robotics. The detection unit is centered on integrated fiber optics, and enables kinetic measures of cellular responses with a time interval of ˜7 or 15 sec. The compound solutions were introduced by using either the on-board liquid handling unit, or the external liquid accessory system; both of which use conventional liquid handling systems. Different RWG biosensor systems including high resolution imaging systems as well as high acquisition optical biosensor systems can also be used.

### G. Biosensors and Biosensor Cellular Assays

Label-free cell-based assays generally employ a biosensor to monitor molecule-induced responses in living cells. The molecule can be naturally occurring or synthetic, and can be a purified or unpurified mixture. A biosensor typically utilizes a transducer such as an optical, electrical, calorimetric, acoustic, magnetic, or like transducer, to convert a molecular recognition event or a molecule-induced change in cells contacted with the biosensor into a quantifiable signal. These label-free biosensors can be used for molecular interaction analysis, which involves characterizing how molecular complexes form and disassociate over time, or for cellular response, which involves characterizing how cells respond to stimulation. The biosensors that are applicable to the present methods can include, for example, optical biosensor systems such as surface plasmon resonance (SPR) and resonant waveguide grating (RWG) biosensors, resonant mirrors, ellipsometers, and electric biosensor systems such as bioimpedance systems.

1. SPR Biosensors and Systems

SPR relies on a prism to direct a wedge of polarized light, covering a range of incident angles, into a planar glass substrate bearing an electrically conducting metallic film (e.g., gold) to excite surface plasmons. The resultant evanescent wave interacts with, and is absorbed by, free electron clouds in the gold layer, generating electron charge density waves (i.e., surface plasmons) and causing a reduction in the intensity of the reflected light. The resonance angle at which this intensity minimum occurs is a function of the refractive index of the solution close to the gold layer on the opposing face of the sensor surface.

2. RWG Biosensors and Systems

An RWG biosensor can include, for example, a substrate (e.g., glass), a waveguide thin film with an embedded grating or periodic structure, and a cell layer. The RWG biosensor utilizes the resonant coupling of light into a waveguide by means of a diffraction grating, leading to total internal reflection at the solution-surface interface, which in turn creates an electromagnetic field at the interface. This electromagnetic field is evanescent in nature, meaning that it decays exponentially from the sensor surface; the distance at which it decays to 1/e of its initial value is known as the penetration depth and is a function of the design of a particular RWG biosensor, but is typically on the order of about 200 nm. This type of biosensor exploits such evanescent wave to characterize ligand-induced alterations of a cell layer at or near the sensor surface.

RWG instruments can be subdivided into systems based on angle-shift or wavelength-shift measurements. In a wavelength-shift measurement, polarized light covering a range of incident wavelengths with a constant angle is used to illuminate the waveguide; light at specific wavelengths is coupled into and propagates along the waveguide. Alternatively, in angle-shift instruments, the sensor is illuminated with monochromatic light and the angle at which the light is resonantly coupled is measured.

The resonance conditions are influenced by the cell layer (e.g., cell confluency, adhesion and status), which is in direct contact with the surface of the biosensor. When a ligand or an analyte interacts with a cellular target (e.g., a GPCR, an ion channel, a kinase) in living cells, any change in local refractive index within the cell layer can be detected as a shift in resonant angle (or wavelength).

The Corning® Epic® system uses RWG biosensors for label-free biochemical or cell-based assays (Corning Inc., Corning, N.Y.). The Epic® System consists of an RWG plate reader and SBS (Society for Biomolecular Screening) standard microtiter plates. The detector system in the plate reader exploits integrated fiber optics to measure the shift in wavelength of the incident light, as a result of ligand-induced changes in the cells. A series of illumination-detection heads are arranged in a linear fashion, so that reflection spectra are collected simultaneously from each well within a column of a 384-well microplate. The whole plate is scanned so that each sensor can be addressed multiple times, and each column is addressed in sequence. The wavelengths of the incident light are collected and used for analysis. A temperature-controlling unit can be included in the instrument to minimize spurious shifts in the incident wavelength due to the temperature fluctuations. The measured response represents an averaged response of a population of cells. Varying features of the systems can be automated, such as sample loading, and can be multiplexed, such as with a 96 or 386 well microtiter plate. Liquid handling is carried out by either on-board liquid handler, or an external liquid handling accessory. Specifically, molecule solutions are directly added or pipetted into the wells of a cell assay plate having cells cultured in the bottom of each well. The cell assay plate contains certain volume of assay buffer solution covering the cells. A simple mixing step by pipetting up and down certain times can also be incorporated into the molecule addition step.

3. Electrical Biosensors and Systems

Electrical biosensors consist of a substrate (e.g., plastic), an electrode, and a cell layer. In this electrical detection method, cells are cultured on small gold electrodes arrayed onto a substrate, and the system's electrical impedance is followed with time. The impedance is a measure of changes in the electrical conductivity of the cell layer. Typically, a small constant voltage at a fixed frequency or varied frequencies is applied to the electrode or electrode array, and the electrical current through the circuit is monitored over time. The ligand-induced change in electrical current provides a measure of cell response. Impedance measurement for whole cell sensing was first realized in 1984. Since then, impedance-based measurements have been applied to study a wide range of cellular events, including cell adhesion and spreading, cell micromotion, cell morphological changes, and cell death. Classical impedance systems suffer from high assay variability due to use of a small detection electrode and a large reference electrode. To overcome this variability, the latest generation of systems, such as the CellKey system (MDS Sciex, South San Francisco, Calif.) and RT-CES (ACEA Biosciences Inc., San Diego, Calif.), utilize an integrated circuit having a microelectrode array.

4. High Spatial Resolution Biosensor Imaging Systems

Optical biosensor imaging systems, including SPR imaging systems, ellipsometry imaging systems, and RWG imaging systems, offer high spatial resolution, and can be used in embodiments of the disclosure. For example, SPR Imager®II (GWC Technologies Inc) uses prism-coupled SPR, and takes SPR measurements at a fixed angle of incidence, and collects the reflected light with a CCD camera. Changes on the surface are recorded as reflectivity changes. Thus, SPR imaging collects measurements for all elements of an array simultaneously.

A swept wavelength optical interrogation system based on RWG biosensor for imaging-based application can be employed. In this system, a fast tunable laser source is used to illuminate a sensor or an array of RWG biosensors in a microplate format. The sensor spectrum can be constructed by detecting the optical power reflected from the sensor as a function of time as the laser wavelength scans, and analysis of the measured data with computerized resonant wavelength interrogation modeling results in the construction of spatially resolved images of biosensors having immobilized receptors or a cell layer. The use of an image sensor naturally leads to an imaging based interrogation scheme. 2 dimensional label-free images can be obtained without moving parts.

Alternatively, angular interrogation system with transverse magnetic or p-polarized TM₀ mode can also be used. This system consists of a launch system for generating an array of light beams such that each illuminates a RWG sensor with a dimension of approximately 200 μm×3000 μm or 200 μm×2000 μm, and a CCD camera-based receive system for recording changes in the angles of the light beams reflected from these sensors. The arrayed light beams are obtained by means of a beam splitter in combination with diffractive optical lenses. This system allows up to 49 sensors (in a 7×7 well sensor array) to be simultaneously sampled at every 3 seconds, or up to the whole 384 well microplate to be simultaneously sampled at every 10 seconds.

Alternatively, a scanning wavelength interrogation system can also be used. In this system, a polarized light covering a range of incident wavelengths with a constant angle is used to illuminate and scan across a waveguide grating biosensor, and the reflected light at each location can be recorded simultaneously. Through scanning, a high resolution image across a biosensor can also be achieved.

5. Dynamic Mass Redistribution (DMR) Signals in Living Cells

The cellular response to stimulation through a cellular target can be encoded by the spatial and temporal dynamics of downstream signaling networks. For this reason, monitoring the integration of cell signaling in real time can provide physiologically relevant information that is useful in understanding cell biology and physiology.

Optical biosensors including resonant waveguide grating (RWG) biosensors, can detect an integrated cellular response related to dynamic redistribution of cellular matters, thus providing a non-invasive means for studying cell signaling. All optical biosensors are common in that they can measure changes in local refractive index at or very near the sensor surface. In principle, almost all optical biosensors are applicable for cell sensing, as they can employ an evanescent wave to characterize ligand-induced change in cells. The evanescent-wave is an electromagnetic field, created by the total internal reflection of light at a solution-surface interface, which typically extends a short distance (hundreds of nanometers) into the solution at a characteristic depth known as the penetration depth or sensing volume.

Recently, theoretical and mathematical models have been developed that describe the parameters and nature of optical signals measured in living cells in response to stimulation with ligands. These models, based on a 3-layer waveguide system in combination with known cellular biophysics, link the ligand-induced optical signals to specific cellular processes mediated through a receptor.

Because biosensors measure the average response of the cells located at the area illuminated by the incident light, a highly confluent layer of cells can be used to achieve optimal assay results. Due to the large dimension of the cells as compared to the short penetration depth of a biosensor, the sensor configuration is considered as a non-conventional three-layer system: a substrate, a waveguide film with a grating structure, and a cell layer. Thus, a ligand-induced change in effective refractive index (i.e., the detected signal) can be, to first order, directly proportional to the change in refractive index of the bottom portion of the cell layer:

ΔN=S(C)Δn_{c}

where S(C) is the sensitivity to the cell layer, and Δn_{c} the ligand-induced change in local refractive index of the cell layer sensed by the biosensor. Because the refractive index of a given volume within a cell is largely determined by the concentrations of bio-molecules such as proteins, Δn_{c} can be assumed to be directly proportional to ligand-induced change in local concentrations of cellular targets or molecular assemblies within the sensing volume. Considering the exponentially decaying nature of the evanescent wave extending away from the sensor surface, the ligand-induced optical signal is governed by:

$$Δ N = S ( C ) α d {\sum }_{i} Δ {C}_{i} [ {e}^{\frac{- {z}_{i}}{Δ {Z}_{C}}} - {e}^{\frac{- {z}_{i + 1}}{Δ {Z}_{C}}} ]$$

where ΔZ_{c} is the penetration depth into the cell layer, α the specific refraction increment (about 0.18/mL/g for proteins), zᵢ the distance where the mass redistribution occurs, and d an imaginary thickness of a slice within the cell layer. Here the cell layer is divided into an equal-spaced slice in the vertical direction. The equation above indicates that the ligand-induced optical signal is a sum of mass redistribution occurring at distinct distances away from the sensor surface, each with an unequal contribution to the overall response. Furthermore, the detected signal, in terms of wavelength or angular shifts, is primarily sensitive to mass redistribution occurring perpendicular to the sensor surface. Because of its dynamic nature, it also is referred to as dynamic mass redistribution (DMR) signal.

6. Cells and Biosensors

Cells rely on multiple cellular pathways or machineries to process, encode and integrate the information they receive. Unlike the affinity analysis with optical biosensors that specifically measures the binding of analytes to a protein target, living cells are much more complex and dynamic.

To study cell signaling, cells can be brought in contact with the surface of a biosensor, which can be achieved through cell culture. These cultured cells can be attached onto the biosensor surface through three types of contacts: focal contacts, close contacts and extracellular matrix contacts, each with its own characteristic separation distance from the surface. As a result, the basal cell membranes are generally located away from the surface by ˜10-100 nm. For suspension cells, the cells can be brought in contact with the biosensor surface through either covalent coupling of cell surface receptors, or specific binding of cell surface receptors, or simply settlement by gravity force. For this reason, biosensors are able to sense the bottom portion of cells.

Cells, in many cases, exhibit surface-dependent adhesion and proliferation. In order to achieve robust cell assays, the biosensor surface can require a coating to enhance cell adhesion and proliferation. However, the surface properties can have a direct impact on cell biology. For example, surface-bound ligands can influence the response of cells, as can the mechanical compliance of a substrate material, which dictates how it will deform under forces applied by the cell. Due to differing culture conditions (time, serum concentration, confluency, etc.), the cellular status obtained can be distinct from one surface to another, and from one condition to another. Thus, special efforts to control cellular status can be necessary in order to develop biosensor-based cell assays.

Cells are dynamic objects with relatively large dimensions—typically in the range of tens of microns. Even without stimulation, cells constantly undergo micromotion—a dynamic movement and remodeling of cellular structure, as observed in tissue culture by time lapse microscopy at the sub-cellular resolution, as well as by bio-impedance measurements at the nanometer level.

Under un-stimulated conditions cells generally produce an almost net-zero DMR response as examined with a RWG biosensor. This is partly because of the low spatial resolution of optical biosensors, as determined by the large size of the laser spot and the long propagation length of the coupled light. The size of the laser spot determines the size of the area studied—and usually only one analysis point can be tracked at a time. Thus, the biosensor typically measures an averaged response of a large population of cells located at the light incident area. Although cells undergo micromotion at the single cell level, the large populations of cells give rise to an average net-zero DMR response. Furthermore, intracellular macromolecules are highly organized and spatially restricted to appropriate sites in mammalian cells. The tightly controlled localization of proteins on and within cells determines specific cell functions and responses because the localization allows cells to regulate the specificity and efficiency of proteins interacting with their proper partners and to spatially separate protein activation and deactivation mechanisms. Because of this control, under un-stimulated conditions, the local mass density of cells within the sensing volume can reach an equilibrium state, thus leading to a net-zero optical response. In order to achieve a consistent optical response, the cells examined can be cultured under conventional culture conditions for a period of time such that most of the cells have just completed a single cycle of division.

Living cells have exquisite abilities to sense and respond to exogenous signals. Cell signaling was previously thought to function via linear routes where an environmental cue would trigger a linear chain of reactions resulting in a single well-defined response. However, research has shown that cellular responses to external stimuli are much more complicated. It has become apparent that the information that cells receive can be processed and encoded into complex temporal and spatial patterns of phosphorylation and topological relocation of signaling proteins. The spatial and temporal targeting of proteins to appropriate sites can be crucial to regulating the specificity and efficiency of protein-protein interactions, thus dictating the timing and intensity of cell signaling and responses. Pivotal cellular decisions, such as cytoskeletal reorganization, cell cycle checkpoints and apoptosis, depend on the precise temporal control and relative spatial distribution of activated signal-transducers. Thus, cell signaling mediated through a cellular target such as G protein-coupled receptor (GPCR) typically proceeds in an orderly and regulated manner, and consists of a series of spatial and temporal events, many of which lead to changes in local mass density or redistribution in local cellular matters of cells. These changes or redistribution, when occurring within the sensing volume, can be followed directly in real time using optical biosensors.

7. DMR Signal is a Physiological Response of Living Cells

Through comparison with conventional pharmacological approaches for studying receptor biology, it has been shown that when a ligand is specific to a receptor expressed in a cell system, the ligand-induced DMR signal is receptor-specific, dose-dependent and saturate-able. For a great number of G protein-coupled receptor (GPCR) ligands, the efficacies (measured by EC₅₀ values) are found to be almost identical to those measured using conventional methods. In addition, the DMR signals exhibit expected desensitization patterns, as desensitization and re-sensitization is common to all GPCRs. Furthermore, the DMR signal also maintains the fidelity of GPCR ligands, similar to those obtained using conventional technologies. In addition, the biosensor can distinguish full agonists, partial agonists, inverse agonists, antagonists, and allosteric modulators. Taken together, these findings indicate that the DMR is capable of monitoring physiological responses of living cells.

8. DMR Signals Contain Systems Cell Biology Information of Ligand-Receptor Pairs in Living Cells

The stimulation of cells with a ligand leads to a series of spatial and temporal events, non-limiting examples of which include ligand binding, receptor activation, protein recruitment, receptor internalization and recycling, second messenger alternation, cytoskeletal remodeling, gene expression, and cell adhesion changes. Because each cellular event has its own characteristics (e.g., kinetics, duration, amplitude, mass movement), and the biosensor is primarily sensitive to cellular events that involve mass redistribution within the sensing volume, these cellular events can contribute differently to the overall DMR signal. Chemical biology, cell biology and biophysical approaches can be used to elucidate the cellular mechanisms for a ligand-induced DMR signal. Recently, chemical biology, which directly uses chemicals for intervention in a specific cell signaling component, has been used to address biological questions. This is possible due to the identification of a great number of modulators that specifically control the activities of many different types of cellular targets. This approach has been adopted to map the signaling and its network interactions mediated through a receptor, including epidermal growth factor (EGF) receptor, and G_{q}- and Gₛ-coupled receptors.

EGFR belongs to the family of receptor tyrosine kinases. EGF binds to and stimulates the intrinsic protein-tyrosine kinase activity of EGFR, initiating a signal transduction cascade, principally involving the MAPK, Akt and INK pathways. Upon EGF stimulation, there are many events leading to mass redistribution in A431 cells—a cell line endogenously over-expressing EGFRs. It is known that EGFR signaling depends on cellular status. As a result, the EGF-induced DMR signals are also dependent on the cellular status. In quiescent cells obtained through 20 hr culturing in 0.1% fetal bovine serum, EGF stimulation leads to a DMR signal with three distinct and sequential phases: (i) a positive phase with increased signal (P-DMR), (ii) a transition phase, and (iii) a decay phase (N-DMR). Chemical biology and cell biology studies show that the EGF-induced DMR signal is primarily linked to the Ras/MAPK pathway, which proceeds through MEK and leads to cell detachment. Two lines of evidence indicate that the P-DMR is mainly due to the recruitment of intracellular targets to the activated receptors at the cell surface. First, blockage of either dynamin or clathrin activity has little effect on the amplitude of the P-DMR event. Dynamin and clathrin, two downstream components of EGFR activation, play crucial roles in executing EGFR internalization and signaling. Second, the blockage of MEK activity partially attenuates the P-DMR event. MEK is an important component in the MAPK pathway, which first translocates from the cytoplasm to the cell membrane, followed by internalization with the receptors, after EGF stimulation.

On the other hand, the N-DMR event is due to cell detachment and receptor internalization. Fluorescent images show that EGF stimulation leads to a significant number of receptors internalized and cell detachment. It is known that blockage of either receptor internalization or MEK activity prevents cell detachment, and receptor internalization requires both dynamin and clathrin. This indicates that blockage of either dynamin or clathrin activity should inhibit both receptor internalization and cell detachment, while blockage of MEK activity should only inhibit cell detachment, but not receptor internalization. As expected, either dynamin or clathrin inhibitors completely inhibit the EGF-induced N-DMR (˜100%), while MEK inhibitors only partially attenuate the N-DMR (˜80%). Fluorescent images also confirm that blocking the activity of dynamin, but not MEK, impairs the receptor internalization

9. DMR Signals Contain Systems Cell Pharmacology Information of a Ligand Acting on Living Cells

Since the DMR signal is an integrated cellular response consisting of contributions of many cellular events involving dynamic redistribution of cellular matters within the bottom portion of cells, a ligand-induced biosensor signal, such as a DMR signal contains systems cell pharmacology information. It is known that GPCRs often display rich behaviors in cells, and that many ligands can induce operative bias to favor specific portions of the cell machinery and exhibit pathway-biased efficacies. Thus, it is highly possibly that a ligand can have multiple efficacies, depending on how cellular events downstream of the receptor are measured and used as readout(s) for the ligand pharmacology. It is difficult in practice for conventional cell assays, which are mostly pathway-biased and assay only a single signaling event, to systematically represent the signaling potentials of GPCR ligands. However, because label-free biosensors cellular assays do not require prior knowledge of cell signaling, and are pathway-unbiased and pathway-sensitive, these biosensor cellular assays are amenable to studying ligand-selective signaling as well as systems cell pharmacology of any ligands.

10. Biosensor Parameters

A label-free biosensor such as RWG biosensor or bioimpedance biosensor is able to follow in real time ligand-induced cellular response. The non-invasive and manipulation-free biosensor cellular assays do not require prior knowledge of cell signaling. The resultant biosensor signal contains high information relating to receptor signaling and ligand pharmacology. Multi-parameters can be extracted from the kinetic biosensor response of cells upon stimulation. These parameters include, but not limited to, the overall dynamics, phases, signal amplitudes, as well as kinetic parameters including the transition time from one phase to another, and the kinetics of each phase (see Fang, Y., and Ferrie, A. M. (2008) “label-free optical biosensor for ligand-directed functional selectivity acting on β2 adrenoceptor in living cells”. FEBS Lett. 582, 558-564; Fang, Y., et al., (2005) “Characteristics of dynamic mass redistribution of EGF receptor signaling in living cells measured with label free optical biosensors”. Anal. Chem., 77, 5720-5725; Fang, Y., et al., (2006) “Resonant waveguide grating biosensor for living cell sensing”. Biophys. J., 91, 1925-1940).

### H. Definitions

Various embodiments of the disclosure will be described in detail with reference to drawings, if any. Reference to various embodiments does not limit the scope of the disclosure, which is limited only by the scope of the claims attached hereto. Additionally, any examples set forth in this specification are not intended to be limiting and merely set forth some of the many possible embodiments for the claimed invention.

1. A

As used in the specification and the appended claims, the singular forms “a,” “an” and “the” or like terms include plural referents unless the context clearly dictates otherwise. Thus, for example, reference to “a pharmaceutical carrier” includes mixtures of two or more such carriers, and the like.

2. Abbreviations

Abbreviations, which are well known to one of ordinary skill in the art, may be used (e.g., “h” or “hr” for hour or hours, “g” or “gm” for gram(s), “mL” for milliliters, and “rt” for room temperature, “nm” for nanometers, “M” for molar, and like abbreviations).

3. About

About modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

4. Assaying

Assaying, assay, or like terms refers to an analysis to determine a characteristic of a substance, such as a molecule or a cell, such as for example, the presence, absence, quantity, extent, kinetics, dynamics, or type of an a cell's optical or bioimpedance response upon stimulation with one or more exogenous stimuli, such as a ligand or marker. Producing a biosensor signal of a cell's response to a stimulus can be an assay.

5. Assaying the Response

“Assaying the response” or like terms means using a means to characterize the response. For example, if a molecule is brought into contact with a cell, a biosensor can be used to assay the response of the cell upon exposure to the molecule.

6. Agonism and Antagonism Mode

The agonism mode or like terms is the assay wherein the cells are exposed to a molecule to determine the ability of the molecule to trigger biosensor signals such as DMR signals, while the antagonism mode is the assay wherein the cells are exposed to a marker in the presence of a molecule to determine the ability of the molecule to modulate the biosensor signal of cells responding to the marker.

7. Biosensor

Biosensor or like terms refer to a device for the detection of an analyte that combines a biological component with a physicochemical detector component. The biosensor typically consists of three parts: a biological component or element (such as tissue, microorganism, pathogen, cells, or combinations thereof), a detector element (works in a physicochemical way such as optical, piezoelectric, electrochemical, thermometric, or magnetic), and a transducer associated with both components. The biological component or element can be, for example, a living cell, a pathogen, or combinations thereof. In embodiments, an optical biosensor can comprise an optical transducer for converting a molecular recognition or molecular stimulation event in a living cell, a pathogen, or combinations thereof into a quantifiable signal.

8. Biosensor Response

A “biosensor response”, “biosensor output signal”, “biosensor signal” or like terms is any reaction of a sensor system having a cell to a cellular response. A biosensor converts a cellular response to a quantifiable sensor response. A biosensor response is an optical response upon stimulation as measured by an optical biosensor such as RWG or SPR or it is a bioimpedence response of the cells upon stimulation as measured by an electric biosensor. Since a biosensor response is directly associated with the cellular response upon stimulation, the biosensor response and the cellular response can be used interchangeably, in embodiments of disclosure.

9. Biosensor Signal

A “biosensor signal” or like terms refers to the signal of cells measured with a biosensor that is produced by the response of a cell upon stimulation.

10. Cell

Cell or like term refers to a small usually microscopic mass of protoplasm bounded externally by a semipermeable membrane, optionally including one or more nuclei and various other organelles, capable alone or interacting with other like masses of performing all the fundamental functions of life, and forming the smallest structural unit of living matter capable of functioning independently including synthetic cell constructs, cell model systems, and like artificial cellular systems.

A cell can include different cell types, such as a cell associated with a specific disease, a type of cell from a specific origin, a type of cell associated with a specific target, or a type of cell associated with a specific physiological function. A cell can also be a native cell, an engineered cell, a transformed cell, an immortalized cell, a primary cell, an embryonic stem cell, an adult stem cell, a cancer stem cell, or a stem cell derived cell.

Human consists of about 210 known distinct cell types. The numbers of types of cells can almost unlimited, considering how the cells are prepared (e.g., engineered, transformed, immortalized, or freshly isolated from a human body) and where the cells are obtained (e.g., human bodies of different ages or different disease stages, etc).

11. Cell Culture

“Cell culture” or “cell culturing” refers to the process by which either prokaryotic or eukaryotic cells are grown under controlled conditions. “Cell culture” not only refers to the culturing of cells derived from multicellular eukaryotes, especially animal cells, but also the culturing of complex tissues and organs.

12. Cell Panel

A “cell panel” or like terms is a panel which comprises at least two types of cells. The cells can be of any type or combination disclosed herein.

13. Cellular Response

A “cellular response” or like terms is any reaction by the cell to a stimulation.

14. Cellular Process

A cellular process or like terms is a process that takes place in or by a cell. Examples of cellular process include, but not limited to, proliferation, apoptosis, necrosis, differentiation, cell signal transduction, polarity change, migration, or transformation.

15. Cellular Target

A “cellular target” or like terms is a biopolymer such as a protein or nucleic acid whose activity can be modified by an external stimulus. Cellular targets commonly are proteins such as enzymes, kinases, ion channels, and receptors.

16. Characterizing

Characterizing or like terms refers to gathering information about any property of a substance, such as a ligand, molecule, marker, or cell, such as obtaining a profile for the ligand, molecule, marker, or cell.

17. Comprise

Throughout the description and claims of this specification, the word “comprise” and variations of the word, such as “comprising” and “comprises,” means “including but not limited to,” and is not intended to exclude, for example, other additives, components, integers or steps.

18. Consisting Essentially of

“Consisting essentially of” in embodiments refers, for example, to a surface composition, a method of making or using a surface composition, formulation, or composition on the surface of the biosensor, and articles, devices, or apparatus of the disclosure, and can include the components or steps listed in the claim, plus other components or steps that do not materially affect the basic and novel properties of the compositions, articles, apparatus, and methods of making and use of the disclosure, such as particular reactants, particular additives or ingredients, a particular agents, a particular cell or cell line, a particular surface modifier or condition, a particular ligand candidate, or like structure, material, or process variable selected. Items that may materially affect the basic properties of the components or steps of the disclosure or may impart undesirable characteristics to the present disclosure include, for example, decreased affinity of the cell for the biosensor surface, aberrant affinity of a stimulus for a cell surface receptor or for an intracellular receptor, anomalous or contrary cell activity in response to a ligand candidate or like stimulus, and like characteristics.

19. Components

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these molecules may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

20. Contacting

Contacting or like terms means bringing into proximity such that a molecular interaction can take place, if a molecular interaction is possible between at least two things, such as molecules, cells, markers, at least a compound or composition, or at least two compositions, or any of these with an article(s) or with a machine. For example, contacting refers to bringing at least two compositions, molecules, articles, or things into contact, i.e. such that they are in proximity to mix or touch. For example, having a solution of composition A and cultured cell B and pouring solution of composition A over cultured cell B would be bringing solution of composition A in contact with cell culture B. Contacting a cell with a ligand would be bringing a ligand to the cell to ensure the cell have access to the ligand.

It is understood that anything disclosed herein can be brought into contact with anything else. For example, a cell can be brought into contact with a marker or a molecule, a biosensor, and so forth.

21. Compounds and Compositions

Compounds and compositions have their standard meaning in the art. It is understood that wherever, a particular designation, such as a molecule, substance, marker, cell, or reagent compositions comprising, consisting of, and consisting essentially of these designations are disclosed. Thus, where the particular designation marker is used, it is understood that also disclosed would be compositions comprising that marker, consisting of that marker, or consisting essentially of that marker. Where appropriate wherever a particular designation is made, it is understood that the compound of that designation is also disclosed. For example, if particular biological material, such as EGF, is disclosed EGF in its compound form is also disclosed.

22. Control

The terms control or “control levels” or “control cells” or like terms are defined as the standard by which a change is measured, for example, the controls are not subjected to the experiment, but are instead subjected to a defined set of parameters, or the controls are based on pre- or post-treatment levels. They can either be run in parallel with or before or after a test run, or they can be a pre-determined standard. For example, a control can refer to the results from an experiment in which the subjects or objects or reagents etc are treated as in a parallel experiment except for omission of the procedure or agent or variable etc under test and which is used as a standard of comparison in judging experimental effects. Thus, the control can be used to determine the effects related to the procedure or agent or variable etc. For example, if the effect of a test molecule on a cell was in question, one could a) simply record the characteristics of the cell in the presence of the molecule, b) perform a and then also record the effects of adding a control molecule with a known activity or lack of activity, or a control composition (e.g., the assay buffer solution (the vehicle)) and then compare effects of the test molecule to the control. In certain circumstances once a control is performed the control can be used as a standard, in which the control experiment does not have to be performed again and in other circumstances the control experiment should be run in parallel each time a comparison will be made.

23. Detect

Detect or like terms refer to an ability of the apparatus and methods of the disclosure to discover or sense a molecule- or a marker-induced cellular response and to distinguish the sensed responses for distinct molecules.

24. Direct Action (of a Drug Candidate Molecule)

A “direct action” or like terms is a result (of a drug candidate molecule”) acting independently on a cell.

25. DMR Signal

A “DMR signal” or like terms refers to the signal of cells measured with an optical biosensor that is produced by the response of a cell upon stimulation.

26. DMR Response

A “DMR response” or like terms is a biosensor response using an optical biosensor. The DMR refers to dynamic mass redistribution or dynamic cellular matter redistribution. A P-DMR is a positive DMR response, a N-DMR is a negative DMR response, and a RP-DMR is a recovery P-DMR response.

27. Drug Candidate Molecule

A drug candidate molecule or like terms is a test molecule which is being tested for its ability to function as a drug or a pharmacophore. This molecule may be considered as a lead molecule.

28. Efficacy

Efficacy or like terms is the capacity to produce a desired size of an effect under ideal or optimal conditions. It is these conditions that distinguish efficacy from the related concept of effectiveness, which relates to change under real-life conditions. Efficacy is the relationship between receptor occupancy and the ability to initiate a response at the molecular, cellular, tissue or system level.

29. Higher and Inhibit and Like Words

The terms higher, increases, elevates, or elevation or like terms or variants of these terms, refer to increases above basal levels, e.g., as compared a control. The terms low, lower, reduces, decreases or reduction or like terms or variation of these terms, refer to decreases below basal levels, e.g., as compared to a control. For example, basal levels are normal in vivo levels prior to, or in the absence of, or addition of a molecule such as an agonist or antagonist to a cell Inhibit or forms of inhibit or like terms refers to reducing or suppressing.

30. In the Presence of the Molecule

“in the presence of the molecule” or like terms refers to the contact or exposure of the cultured cell with the molecule. The contact or exposure can be taken place before, or at the time, the stimulus is brought to contact with the cell.

31. Index

An index or like terms is a collection of data. For example, an index can be a list, table, file, or catalog that contains one or more modulation profiles. It is understood that an index can be produced from any combination of data. For example, a DMR profile can have a P-DMR, a N-DMR, and a RP-DMR. An index can be produced using the completed date of the profile, the P-DMR data, the N-DMR data, the RP-DMR data, or any point within these, or in combination of these or other data. The index is the collection of any such information. Typically, when comparing indexes, the indexes are of like data, i.e. P-DMR to P-DMR data.

i. Biosensor Index

A “biosensor index” or like terms is an index made up of a collection of biosensor data. A biosensor index can be a collection of biosensor profiles, such as primary profiles, or secondary profiles. The index can be comprised of any type of data. For example, an index of profiles could be comprised of just an N-DMR data point, it could be a P-DMR data point, or both or it could be an impedence data point. It could be all of the data points associated with the profile curve.

ii. DMR Index

A “DMR index” or like terms is a biosensor index made up of a collection of DMR data.

32. Known Molecule

A known molecule or like terms is a molecule with known pharmacological/biological/physiological/pathophysiological activity whose precise mode of action(s) may be known or unknown.

33. Known Modulator

A known modulator or like terms is a modulator where at least one of the targets is known with a known affinity. For example, a known modulator could be a PI3K inhibitor, a PKA inhibitor, a GPCR antagonist, a GPCR agonist, a RTK inhibitor, an epidermal growth factor receptor neutralizing antibody, or a phosphodiesterase inhibition, a PKC inhibitor or activator, etc.

34. Known Modulator Biosensor Index

A “known modulator biosensor index” or like terms is a modulator biosensor index produced by data collected for a known modulator. For example, a known modulator biosensor index can be made up of a profile of the known modulator acting on the panel of cells, and the modulation profile of the known modulator against the panels of markers, each panel of markers for a cell in the panel of cells.

35. Known Modulator DMR Index

A “known modulator DMR index” or like terms is a modulator DMR index produced by data collected for a known modulator. For example, a known modulator DMR index can be made up of a profile of the known modulator acting on the panel of cells, and the modulation profile of the known modulator against the panels of markers, each panel of markers for a cell in the panel of cells.

36. Ligand

A ligand or like terms is a substance or a composition or a molecule that is able to bind to and form a complex with a biomolecule to serve a biological purpose. Actual irreversible covalent binding between a ligand and its target molecule is rare in biological systems. Ligand binding to receptors alters the chemical conformation, i.e., the three dimensional shape of the receptor protein. The conformational state of a receptor protein determines the functional state of the receptor. The tendency or strength of binding is called affinity Ligands include substrates, blockers, inhibitors, activators, and neurotransmitters. Radioligands are radioisotope labeled ligands, while fluorescent ligands are fluorescently tagged ligands; both can be considered as ligands are often used as tracers for receptor biology and biochemistry studies. Ligand and modulator are used interchangeably.

37. Library

A library or like terms is a collection. The library can be a collection of anything disclosed herein. For example, it can be a collection, of indexes, an index library; it can be a collection of profiles, a profile library; or it can be a collection of DMR indexes, a DMR index library; Also, it can be a collection of molecule, a molecule library; it can be a collection of cells, a cell library; it can be a collection of markers, a marker library; a library can be for example, random or non-random, determined or undetermined For example, disclosed are libraries of DMR indexes or biosensor indexes of known modulators.

38. Marker

A marker or like terms is a ligand which produces a signal in a biosensor cellular assay. The signal is, must also be, characteristic of at least one specific cell signaling pathway(s) and/or at least one specific cellular process(es) mediated through at least one specific target(s). The signal can be positive, or negative, or any combinations (e.g., oscillation).

39. Marker Panel

A “marker panel” or like terms is a panel which comprises at least two markers. The markers can be for different pathways, the same pathway, different targets, or even the same targets.

40. Marker Biosensor Index

A “marker biosensor index” or like terms is a biosensor index produced by data collected for a marker. For example, a marker biosensor index can be made up of a profile of the marker acting on the panel of cells, and the modulation profile of the marker against the panels of markers, each panel of markers for a cell in the panel of cells.

41. Marker DMR index

A “marker biosensor index” or like terms is a biosensor DMR index produced by data collected for a marker. For example, a marker DMR index can be made up of a profile of the marker acting on the panel of cells, and the modulation profile of the marker against the panels of markers, each panel of markers for a cell in the panel of cells.

42. Material

Material is the tangible part of something (chemical, biochemical, biological, or mixed) that goes into the makeup of a physical object.

43. Mimic

As used herein, “mimic” or like terms refers to performing one or more of the functions of a reference object. For example, a molecule mimic performs one or more of the functions of a molecule.

44. Modulate

To modulate, or forms thereof, means either increasing, decreasing, or maintaining a cellular activity mediated through a cellular target. It is understood that wherever one of these words is used it is also disclosed that it could be 1%, 5%, 10%, 20%, 50%, 100%, 500%, or 1000% increased from a control, or it could be 1%, 5%, 10%, 20%, 50%, or 100% decreased from a control.

45. Modulator

A modulator or like terms is a ligand that controls the activity of a cellular target. It is a signal modulating molecule binding to a cellular target, such as a target protein.

46. Modulation Comparison

A “modulation comparison” or like terms is a result of normalizing a primary profile and a secondary profile.

47. Modulator Biosensor Index

A “modulator biosensor index” or like terms is a biosensor index produced by data collected for a modulator. For example, a modulator biosensor index can be made up of a profile of the modulator acting on the panel of cells, and the modulation profile of the modulator against the panels of markers, each panel of markers for a cell in the panel of cells.

48. Modulator DMR Index

A “modulator DMR index” or like terms is a DMR index produced by data collected for a modulator. For example, a modulator DMR index can be made up of a profile of the modulator acting on the panel of cells, and the modulation profile of the modulator against the panels of markers, each panel of markers for a cell in the panel of cells.

49. Modulate the Biosensor Signal of a Marker

Modulate the biosensor signal or like terms is to cause changes of the biosensor signal or profile of a cell in response to stimulation with a marker.

50. Modulate the DMR Signal

Modulate the DMR signal or like terms is to cause changes of the DMR signal or profile of a cell in response to stimulation with a marker.

51. Molecule

As used herein, the terms “molecule” or like terms refers to a biological or biochemical or chemical entity that exists in the form of a chemical molecule or molecule with a definite molecular weight. A molecule or like terms is a chemical, biochemical or biological molecule, regardless of its size.

Many molecules are of the type referred to as organic molecules (molecules containing carbon atoms, among others, connected by covalent bonds), although some molecules do not contain carbon (including simple molecular gases such as molecular oxygen and more complex molecules such as some sulfur-based polymers). The general term “molecule” includes numerous descriptive classes or groups of molecules, such as proteins, nucleic acids, carbohydrates, steroids, organic pharmaceuticals, small molecule, receptors, antibodies, and lipids. When appropriate, one or more of these more descriptive terms (many of which, such as “protein,” themselves describe overlapping groups of molecules) will be used herein because of application of the method to a subgroup of molecules, without detracting from the intent to have such molecules be representative of both the general class “molecules” and the named subclass, such as proteins. Unless specifically indicated, the word “molecule” would include the specific molecule and salts thereof, such as pharmaceutically acceptable salts.

52. Molecule Mixture

A molecule mixture or like terms is a mixture containing at least two molecules. The two molecules can be, but not limited to, structurally different (i.e., enantiomers), or compositionally different (e.g., protein isoforms, glycoform, or an antibody with different poly(ethylene glycol) (PEG) modifications), or structurally and compositionally different (e.g., unpurified natural extracts, or unpurified synthetic compounds).

53. Molecule Biosensor Index

A “molecule biosensor index” or like terms is a biosensor index produced by data collected for a molecule. For example, a molecule biosensor index can be made up of a profile of the molecule acting on the panel of cells, and the modulation profile of the molecule against the panels of markers, each panel of markers for a cell in the panel of cells.

54. Molecule DMR Index

A “molecule DMR index” or like terms is a DMR index produced by data collected for a molecule. For example, a molecule biosensor index can be made up of a profile of the molecule acting on the panel of cells, and the modulation profile of the molecule against the panels of markers, each panel of markers for a cell in the panel of cells.

55. Molecule Index

A “molecule index” or like terms is an index related to the molecule.

56. Molecule-Treated Cell

A molecule-treated cell or like terms is a cell that has been exposed to a molecule.

57. Molecule Modulation Index

A “molecule modulation index” or like terms is an index to display the ability of the molecule to modulate the biosensor output signals of the panels of markers acting on the panel of cells. The modulation index is generated by normalizing a specific biosensor output signal parameter of a response of a cell upon stimulation with a marker in the presence of a molecule against that in the absence of any molecule.

58. Molecule Pharmacology

Molecule pharmacology or the like terms refers to the systems cell biology or systems cell pharmacology or mode(s) of action of a molecule acting on a cell. The molecule pharmacology is often characterized by, but not limited, toxicity, ability to influence specific cellular process(es) (e.g., proliferation, differentiation, reactive oxygen species signaling), or ability to modulate a specific cellular target (e.g, PI3K, PKA, PKC, PKG, JAK2, MAPK, MEK2, or actin).

59. Normalizing

Normalizing or like terms means, adjusting data, or a profile, or a response, for example, to remove at least one common variable. For example, if two responses are generated, one for a marker acting a cell and one for a marker and molecule acting on the cell, normalizing would refer to the action of comparing the marker-induced response in the absence of the molecule and the response in the presence of the molecule, and removing the response due to the marker only, such that the normalized response would represent the response due to the modulation of the molecule against the marker. A modulation comparison is produced by normalizing a primary profile of the marker and a secondary profile of the marker in the presence of a molecule (modulation profile).

60. Optional

“Optional” or “optionally” or like terms means that the subsequently described event or circumstance can or cannot occur, and that the description includes instances where the event or circumstance occurs and instances where it does not. For example, the phrase “optionally the composition can comprise a combination” means that the composition may comprise a combination of different molecules or may not include a combination such that the description includes both the combination and the absence of the combination (i.e., individual members of the combination).

61. Or

The word “or” or like terms as used herein means any one member of a particular list and also includes any combination of members of that list.

62. Profile

A profile or like terms refers to the data which is collected for a composition, such as a cell. A profile can be collected from a label free biosensor as described herein.

i. Primary Profile

A “primary profile” or like terms refers to a biosensor response or biosensor output signal or profile which is produced when a molecule contacts a cell. Typically, the primary profile is obtained after normalization of initial cellular response to the net-zero biosensor signal (i.e., baseline)

ii. Secondary Profile

A “secondary profile” or like terms is a biosensor response or biosensor output signal of cells in response to a marker in the presence of a molecule. A secondary profile can be used as an indicator of the ability of the molecule to modulate the marker-induced cellular response or biosensor response.

iii. Modulation Profile

A “modulation profile” or like terms is the comparison between a secondary profile of the marker in the presence of a molecule and the primary profile of the marker in the absence of any molecule. The comparison can be by, for example, subtracting the primary profile from secondary profile or subtracting the secondary profile from the primary profile or normalizing the secondary profile against the primary profile.

63. Panel

A panel or like terms is a predetermined set of specimens (e.g., markers, or cells, or pathways). A panel can be produced from picking specimens from a library.

64. Positive Control

A “positive control” or like terms is a control that shows that the conditions for data collection can lead to data collection.

65. Potentiate

Potentiate, potentiated or like terms refers to an increase of a specific parameter of a biosensor response of a marker in a cell caused by a molecule. By comparing the primary profile of a marker with the secondary profile of the same marker in the same cell in the presence of a molecule, one can calculate the modulation of the marker-induced biosensor response of the cells by the molecule. A positive modulation means the molecule to cause increase in the biosensor signal induced by the marker.

66. Potency

Potency or like terms is a measure of molecule activity expressed in terms of the amount required to produce an effect of given intensity. For example, a highly potent drug evokes a larger response at low concentrations. The potency is proportional to affinity and efficacy. Affinity is the ability of the drug molecule to bind to a receptor.

67. Publications

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

68. Receptor

A receptor or like terms is a protein molecule embedded in either the plasma membrane or cytoplasm of a cell, to which a mobile signaling (or “signal”) molecule may attach. A molecule which binds to a receptor is called a “ligand,” and may be a peptide (such as a neurotransmitter), a hormone, a pharmaceutical drug, or a toxin, and when such binding occurs, the receptor goes into a conformational change which ordinarily initiates a cellular response. However, some ligands merely block receptors without inducing any response (e.g. antagonists). Ligand-induced changes in receptors result in physiological changes which constitute the biological activity of the ligands.

69. “Robust Biosensor Signal”

A “robust biosensor signal” is a biosensor signal whose amplitude(s) is significantly (such as 3×, 10×, 20×, 100×, or 1000×) above either the noise level, or the negative control response. The negative control response is often the biosensor response of cells after addition of the assay buffer solution (i.e., the vehicle). The noise level is the biosensor signal of cells without further addition of any solution. It is worthy of noting that the cells are always covered with a solution before addition of any solution.

70. “Robust DMR Signal”

A “robust DMR signal” or like terms is a DMR form of a “robust biosensor signal.”

71. Ranges

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

72. Response

A response or like terms is any reaction to any stimulation.

73. Sample

By sample or like terms is meant an animal, a plant, a fungus, etc.; a natural product, a natural product extract, etc.; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

74. Signaling Pathway(s)

A “defined pathway” or like terms is a path of a cell from receiving a signal (e.g., an exogenous ligand) to a cellular response (e.g., increased expression of a cellular target). In some cases, receptor activation caused by ligand binding to a receptor is directly coupled to the cell's response to the ligand. For example, the neurotransmitter GABA can activate a cell surface receptor that is part of an ion channel. GABA binding to a GABA A receptor on a neuron opens a chloride-selective ion channel that is part of the receptor. GABA A receptor activation allows negatively charged chloride ions to move into the neuron which inhibits the ability of the neuron to produce action potentials. However, for many cell surface receptors, ligand-receptor interactions are not directly linked to the cell's response. The activated receptor must first interact with other proteins inside the cell before the ultimate physiological effect of the ligand on the cell's behavior is produced. Often, the behavior of a chain of several interacting cell proteins is altered following receptor activation. The entire set of cell changes induced by receptor activation is called a signal transduction mechanism or pathway. The signaling pathway can be either relatively simple or quite complicated.

75. Similarity of Indexes

“Similarity of indexes” or like terms is a term to express the similarity between two indexes, or among at least three indices, one for a molecule, based on the patterns of indices, and/or a matrix of scores. The matrix of scores are strongly related to their counterparts, such as the signatures of the primary profiles of different molecules in corresponding cells, and the nature and percentages of the modulation profiles of different molecules against each marker. For example, higher scores are given to more-similar characters, and lower or negative scores for dissimilar characters. Because there are only three types of modulation, positive, negative and neutral, found in the molecule modulation index, the similarity matrices are relatively simple. For example, a simple matrix will assign identical modulation (e.g., a positive modulation) a score of +1 and non-identical modulation a score of −1.

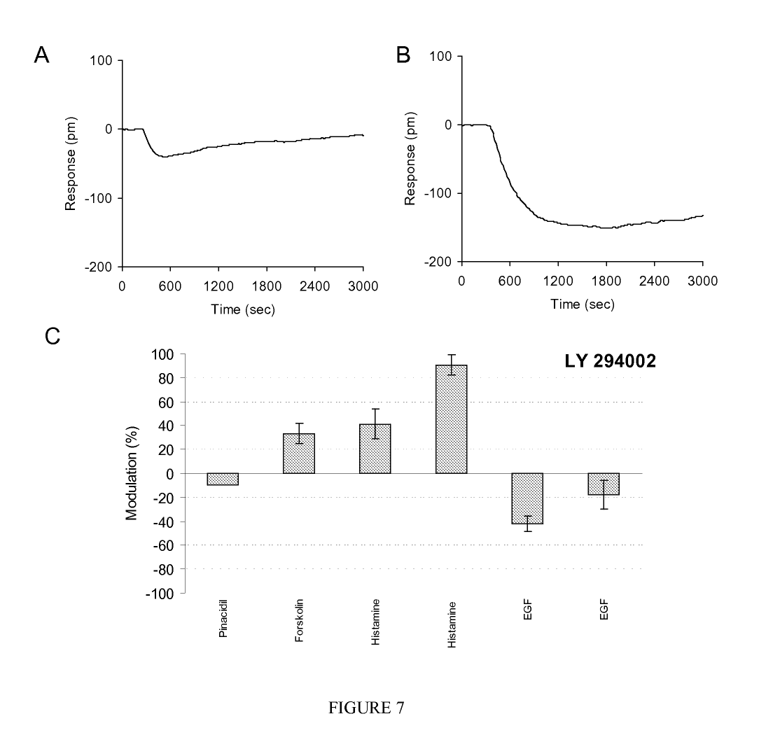

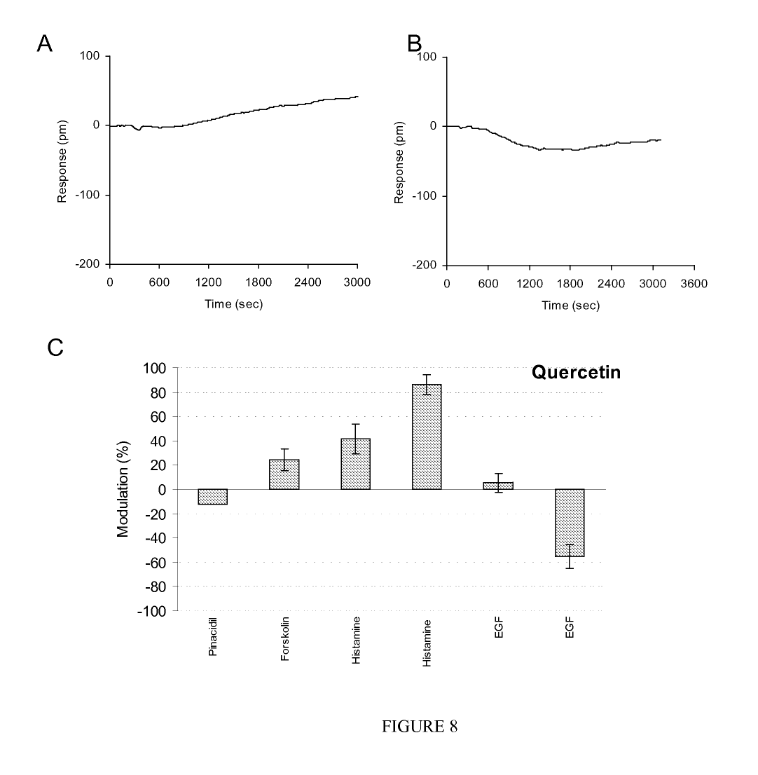

Alternatively, different scores can be given for a type of modulation but with different scales. For example, a positive modulation of 10%, 20%, 30%, 40%, 50%, 60%, 100%, 200%, etc, can be given a score of +1, +2, +3, +4, +5, +6, +10, +20, correspondingly. Conversely, for negative modulation, similar but in opposite score can be given. Following this approach, the modulation index of LY294002 against panels of markers, as shown in FIG. 7C, illustrates that the non-selective PI3K inhibitor LY294002 modulates differently the biosensor response induced by different markers: pinacidil (−9%), forskolin (+33%), histamine (the early P-DMR, +41%; the late P-DMR, +90%), all in A549 cell; and EGF (P-DMR, −42%), and EGF (N-DMR, −18%), in quiescent A431 cells. Thus, the score of LY294002 modulation index in coordination can be assigned as (−1, 3, 4, 9, −4, −2). Similarly, for quercetin shown in FIG. 8C, its score in coordination is (−1, 2, 4, 9, 0, −5). By comparing the scores between LY294002 and quercetin, one can conclude that both molecules exhibits similar mode(s) of action, i.e., non-selective Pi3K inhibitor, in the two cell lines examined.

76. Stable

When used with respect to pharmaceutical compositions, the term “stable” or like terms is generally understood in the art as meaning less than a certain amount, usually 10%, loss of the active ingredient under specified storage conditions for a stated period of time. The time required for a composition to be considered stable is relative to the use of each product and is dictated by the commercial practicalities of producing the product, holding it for quality control and inspection, shipping it to a wholesaler or direct to a customer where it is held again in storage before its eventual use. Including a safety factor of a few months time, the minimum product life for pharmaceuticals is usually one year, and preferably more than 18 months. As used herein, the term “stable” references these market realities and the ability to store and transport the product at readily attainable environmental conditions such as refrigerated conditions, 2° C. to 8° C.

77. Substance

A substance or like terms is any physical object. A material is a substance. Molecules, ligands, markers, cells, proteins, and DNA can be considered substances. A machine or an article would be considered to be made of substances, rather than considered a substance themselves.

78. Subject

As used throughout, by a subject or like terms is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) and mammals, non-human mammals, primates, non-human primates, rodents, birds, reptiles, amphibians, fish, and any other animal. In one aspect, the subject is a mammal such as a primate or a human. The subject can be a non-human.

79. Test Molecule

A test molecule or like terms is a molecule which is used in a method to gain some information about the test molecule. A test molecule can be an unknown or a known molecule.

80. Treating

Treating or treatment or like terms can be used in at least two ways. First, treating or treatment or like terms can refer to administration or action taken towards a subject. Second, treating or treatment or like terms can refer to mixing any two things together, such as any two or more substances together, such as a molecule and a cell. This mixing will bring the at least two substances together such that a contact between them can take place.

When treating or treatment or like terms is used in the context of a subject with a disease, it does not imply a cure or even a reduction of a symptom for example. When the term therapeutic or like terms is used in conjunction with treating or treatment or like terms, it means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

81. Trigger

A trigger or like terms refers to the act of setting off or initiating an event, such as a response.

82. Values

Specific and preferred values disclosed for components, ingredients, additives, cell types, markers, and like aspects, and ranges thereof, are for illustration only; they do not exclude other defined values or other values within defined ranges. The compositions, apparatus, and methods of the disclosure include those having any value or any combination of the values, specific values, more specific values, and preferred values described herein.

Thus, the disclosed methods, compositions, articles, and machines, can be combined in a manner to comprise, consist of, or consist essentially of, the various components, steps, molecules, and composition, and the like, discussed herein. They can be used, for example, in methods for characterizing a molecule including a ligand as defined herein; a method of producing an index as defined herein; or a method of drug discovery as defined herein.

83. Unknown Molecule

An unknown molecule or like terms is a molecule with unknown biological/pharmacological/physiological/pathophysiological activity.

84. Pathway

A pathway as used herein is a series of chemical reactions occurring within a cell. Within a pathway, one molecule or substance is modified which then leads to another molecule or substance being modified and so on. Often, enzymes such as kinases are involved in these modifications. Pathways can occur from the cell surface to the nucleus, as well as from organelle to organelle within a cell, or from cytosol to organelle or organelle to cytosol. The modification includes chemical (e.g., phosphorylation) or physical (e.g., translocation from one location to another) modifications.

85. Signaling

Signaling refers to the modification of one molecule or substance in a pathway leading to another modification of another molecule or substance within a pathway.

86. Deregulated PI3K Pathway Cell Line

A deregulated PI3K pathway cell line is a cell line in which the PI3K pathway is hyperactivated, due to alteration of crucial signaling cascade protein(s) in the pathway. These alterations include, but not limited to, mutations of K-Ras (a upstream protein of PI3K) which lead to constitutive activation of Ras and PI3K in the unstimulated cells, or loss-of-function of PTEN (a downstream negative regulator of PI3K) which also lead to constitutive activation of PI3K. For example, a deregulated PI3K pathway cell line is the A549 cell line which contain a K-Ras mutant that is constitutively activated in the unstimulated cells (Krypuy, M., et al. “High resolution melting analysis for the rapid and sensitive detection of mutations in clinical samples: KRAS codon 12 and 13 mutations in non-small cell lung cancer”. BMC Cancer 2006, 6, e295).

Proteins involved in the PI3K pathway include, but not limited to, (1) AKT and PI3K family members and their Regulators: AKT1, AKT2, AKT3, APPL, BTK, CTMP, GNB1, GRB10, GRB2, HSPB1, HSPCA, HSPCB, ILK, INPP5D (SHIP), INPPL1, MAPK8IP1 (JIP1), MTCP1, PDK2, PDPK1, PIK3CA (p110a), PIK3CB (p110b), PIK3CG, PIK3R1 (p85a), PIK3R2 (p85b), PIK3R3, PRKCA, PRKCB1, PRKCZ, PTEN, TCL1A, TCL1B; (2) IGF-1 or other RTK signaling pathway: CSNK2A1, ELK1, FOS, GRB2, HRAS, IGF1, IGF1R, IRS1, JUN, MAP2K1, MAPK3, MAPK8, PIK3CB, PTPN11, RAF1, KRAS, RASA1, SHC1, SOS1, SRF; (3) PI3K subunit p85-related regulation of actin organization and cell Migration: AICDA, CDC42, CUTL1, PAK1, PDGFRA, RAC1, RHOA, WASL, ZFYVE21; (4) PTEN dependent cell cycle arrest and apoptosis: AKT1, CDKN1B (p27), CUTL1, FASLG, FOXO3A, GRB2, ILK, ITGB1, MAPK1, MAPK3, PDK1, PDK2, PTEN, PTK2, RBL2, SHC1, SOS1, ZFYVE21; (5) BAD phosphorylation-related anti-apoptotic pathways: AKT1, ASAH1, BAD, CUTL1, GRB2, HRAS, IGF1R, IRS1, MAP2K1, MAPK1, MAPK3, PRKAR1B, RAF1, RPS6KA1, SHC1, SOS1, YWHAH, ZFYVE21; and (6) proteins involved in the mTOR signaling pathway: AKT1, CUTL1, EIF3S10, EIF4A1, EIF4B, EIF4E, EIF4EBP1, EIF4G1, FKBP1A, FRAP1, MKNK1, PDK1, PDK2, PR48, PTEN, RHEB, RPS6, RPS6 KB1, TSC1, TSC2, ZFYVE21.

87. data Output

A data output refers to the collected result occurring after performing an assay using an analytical machine, such as a label free biosensor. For example, the data output of a label free biosensor could be a DMR signal. It is understood that data output can be manipulated, for example, into an Index. It is also understood that there can be any kind of data output that the assay is performed with, such as a molecule, PI3K, Rho Kinase, Marker, inhibitor, PI3K inhibitor, marker-molecule, marker-PI3K inhibitor, etc. It is also understood that any two outputs can be compared, such as a molecule data output and a PI3K inhibitor data output forming a molecule-PI3K inhibitor comparison. Typically, such a comparison will be performed with analogous data outputs, such as a DMR data output to a DMR data output.

88. Potential PI3K Inhibitor

A potential PI3K inhibitor is any molecule in which the molecule is determined to be similar to a known PI3K inhibitor as discussed herein. The known PI3K inhibitors include, but not limited to, non-selective PI3K inhibitors such as LY294002, quercetin, and PI-103, and other isoform selective PI3K inhibitors such as PI3Kα, PI3Kβ, PI3Kγ, and PI3Kδ inhibitors. A known PI3K inhibitor can be used as a referencing molecule for comparison.

89. Potential Rho Kinase Inhibitor

A potential Rho kinase (ROCK) inhibitor is any molecule in which the molecule is determined to be similar to a known ROCK inhibitor as discussed herein. The known ROCK inhibitors include, but not limited to, Y27632, H-89, and H-8.

90. K-Ras Activating Mutant Cell Line

A K-Ras activating mutant cell line is any cell line in which there is a K-Ras mutation that is constitutively activated. An example of a K-Ras activating mutant cell line is the A549 cell line.

91. PI3K Inhibitor and Known PI3K Inhibitor

A PI3K inhibitor is any molecule which has been determined to be an inhibitor of PI3K. The known PI3K inhibitors include, but not limited to, non-selective and irreversible PI3K inhibitor wortmannin, non-selective and reversible PI3K inhibitors such as LY294002, quercetin, and PI-103, and other isoform selective PI3K inhibitors such as PI3Kα, PI3Kβ, PI3Kγ, and PI3Kδ inhibitors. A known PI3K inhibitor can be used as a referencing molecule for comparison

92. A Normal PI3K Pathway Cell Line

A normal PI3K pathway cell line is a cell line in which the PI3K pathway is not deregulated, and thus not constitutively activated. However, such cell line may still contain certain protein mutants that are not able to result in constitutive activation of the PI3K pathway.

93. Agonist

An agonist is a molecule or substance that produces an action, such as a molecule binding a receptor on a cell producing a response by the cell, which can be intracellular.

94. Antagonist

An antagonist is a molecule or substance that inhibits, such as blocks, an action, such as the action of an agonist, such as a molecule binding a receptor which prevents an agonist from binding, and thereby inhibits the action of the agonist.

95. Activator

An activator is anything that causes an increase in a state, relative to a basal state. For example, EGF is an activator of an EGFR, and the binding of EGF to EGFR causes signaling event.

96. TLR9 Agonist

A TLR9 agonist is an agonist for the Toll-like receptor subtype 9 (TLR9). The TLR9 agonist can be, such as ODN2006. TLRs are a family of 10 pattern recognition receptors (TLR1, 2, 3, 4, 5, 6, 7, 8, 9 and 10). CpG ODNs are synthetic ODNs that contain unmethylated CpG dinucleotides in particular sequence contexts (CpG motifs). CpG ODNs are recognized by TLR9 leading to strong immunostimulatory effects. TLR9 agonists can be classified into three classes, Type A, B, and C, respectively. Type A ODNs contain a phosphorothioate 3′ poly-G string, inducing high IFN-alpha production from plasmacytoid dendritic cells but are weak in activating NF-kB signaling. Type B ODNs contain a full phosphorothioate backbone with one or more CpGs, activating B cells but weak in IFN-alpha secretion. Type C ODNs contain a complete phosphorothioate backbone and a CpG-containing palindromic motif, inducing IFN-alpha production and B cell activation (Krug, A. et al. (2001) Identification of CpG oligonucleotide sequences with high induction of IFN-alpha/beta in plasmacytoid dendritic cells. Eur. J. Immunol. 31, 2154-2163).

97. Toll-Like Receptors (TLRs)

TLRs are a key component of human innate immunity that senses and combats microbial infection, and also engage multiple mechanisms that control the subsequent shaping of adaptive immune responses. TLRs represent new therapeutic targets for diseases, including infectious diseases, autoimmune disorders, cancer, and allergic diseases.

Toll-like receptors (TLRs) recognize distinct pathogen-associated molecular patterns and play a critical role in innate immune responses. They participate in the first line of defense against invading pathogens and play a significant role in inflammation, immune cell regulation, survival and proliferation. To date 10 members of the TLR family have been identified, of which TLR1, 2, 4, 5 and 6 are located on the cell surface and TLR3, 7, 8 and 9 are localized to the endosomal/lysosomal compartment. The activation of the TLR signaling pathway originates from the cytoplasmic Toll/IL-1 receptor (TIR) domain that associates with a TIR domain-containing adaptor, MyD88. Upon stimulation with ligands, MyD88 recruits IL-1 receptor-associated kinase (IRAK) to TLRs through interaction of the death domains of both molecules. IRAK activated by phosphorylation then associates with TRAF6, finally leading to activation of INK and NF-κB. Tollip and IRAK-M interact with IRAK-1 and negatively regulate the TLR-mediated signaling pathways. MyD88-independent pathways induce activation of IRF3 and expression of interferon-13. TIR-domain containing adaptors such as TIRAP, TRIF and TRAM regulate TLR-mediated signaling pathways by providing specificity for individual TLR signaling cascades.

98. Toll-Like Receptor Cell Line

A Toll-like receptor cell line is a cell line that expresses a toll receptor either endogenously or recombinantly. Thus, there are endogenous toll receptor cell lines and recombinant toll receptor cell lines. For example, the HepG2 cell is a TLR cell line, because it expresses a low level of TLR9, which is located in endosomes and functional. HepG2 also expresses TLR2, TLR3, TLR6, TLR9, and many signaling proteins downstream TLRs (e.g., ICAM1, CD14, MyD88, LY96, TRIF, TICAM2, TIRAP, CD83, SOCS1, TNFAIP3, TOLLIP, IRAK1, IRAK2, IRAK4, TRAF6, CCL5, CXCL10). However, HepG2 does not express TLR1, 4, 5, 7, 8, and 10 (Nishimura, M., and Naito, S. Tissue-specific mRNA expression profiles of human Toll-like receptors and related genes. Biol. Pharm. Bull. 2005, 28, 886-892).

99. ROCK Inhibitor Responsive Cell Line

A ROCK kinase inhibitor responsive cell line is any cell line in which inhibiting the basal activity of ROCKs by a known ROCK inhibitor leads to a detectable biosensor signal in the cell. For example, A549 is also a ROCK inhibitor responsive cell line. The unstimulated A549, due to the presence of activating K-RAS mutants, contains deregulated PI3K/Akt activity. ROCK is a downstream target of PI3K. Thus, inhibiting the basal activity of ROCK by a known ROCK inhibitor such as Y27632 or H89 could lead to a robust DMR signal in A549 cells.

100. IKK Pathway

The IKK pathway refers to any signaling molecule upstream or downstream of IKK or their collection. An agonist for a Toll Receptor, such as poly(IC) can activate the IKK pathway. The IκB kinase (IKK) enzyme complex is part of the upstream NF-κB signal transduction cascade. The IκBα (inhibitor of kappa B) protein inactivates the NF-κB transcription factor by masking the nuclear localization signals (NLS) of NF-κB proteins and keeping them sequestered in an inactive state in the cytoplasm. IKK specifically, phosphorylates the inhibitory IκBα protein. This phosphorylation results in the dissociation of IκBα from NF-κB and thereby activates NF-κB.

101. PI3K/AKT Pathway

The PI3K/AKT pathway refers to any signaling molecule upstream or downstream of AKT or their collection. An agonist for a GPCR, such as the Gq-coupled receptor histamine receptor in A549, can activate the PI3K/AKT pathway. The adenylyl cyclase activator forskolin also leads to activation of the PI3K/AKT pathway via EPAC.

102. Gq-, Gi- and G12/13-Mediated Signaling

Gq-, Gi- and G12/13-mediated signaling refers to any signaling event upstream or downstream of Gq-, Gi- and G12/13. An activator of such signaling can be an agonist, such as SLIGKV-amide, for endogenous protease activated receptor subtype 2 (PAR2).

103. Rho-Mediated Signaling

Rho mediated signaling refers to any signaling upstream or downstream of a molecule or substance which interacts with Rho kinases. An opener for endogenous ATP-sensitive potassium (KATP) ion channel, such as pinacidil, can be an activator of Rho mediated signaling.

104. JAK (Janus Kinase) Mediated Signaling

JAK mediated signaling refers to a signaling upstream or downstream of a molecule or substance which interacts with JAK. An opener for endogenous ATP-sensitive potassium (KATP) ion channel, such as pinacidil, can be an activator of JAK mediated signaling. JAKs and STATs (Signal transduction and transcription proteins) are critical components of many cytokine receptor systems, regulating growth, survival, differentiation and pathogen resistance. An example is the IL-6 (or gp130) family of receptors, which co-regulate B cell differentiation, plasmacytogenesis and the acute phase reaction. Cytokine binding induces receptor dimerization, activating the associated JAKs, which phosphorylate themselves and the receptor. The phosphorylated sites on the receptor and Jaks serve as docking sites for the SH2-containing Stats, such as Stat3, and for SH2-containing proteins and adaptors that link the receptor to MAP kinase, PI3 Kinase/Akt and other cellular pathways.

Janus kinase mutations are major molecular events in human hematological malignancies. A unique somatic mutation in the Jak2 pseudokinase domain (V617F) occurs in >90% of polycythemia vera patients, and in a large proportion of essential thrombocythemia and idiopathic myelofibrosis patients. This mutation results in the pathologic activation Jak2 kinase, which leads to malignant transformation of hematopoietic progenitors. Several Jak3 pseudokinase domain mutations, present in some patients with acute megakaryoblastic leukemia, also render Jak3 constitutively active. Somatic acquired gain-of function mutations in Jak1 have been discovered in approximately 20% of adult T-cell acute lymphoblastic leukemia.

105. PKC Pathway

A PKC pathway is any signaling molecule or substance upstream or downstream of PKC or their collection. An activator of a PKC pathway is Phorbol 12-myristate 13-acetate (PMA), which directly activates PKC and its signaling pathway. An agonist for a Gq-coupled receptor or RTKs can also be used to activate PKC pathway.

106. Histamine Receptors

A histamine receptor is any receptor for which histamine acts as an agonist. Histamine receptors are a family of four G protein-coupled receptors, including H1, H2, H3 and H4 receptors. Histamine receptors are often endogenously expressed in immune cells such as A549 and A431 cells.

107. cAMP-PKA Pathway

The cAMP-PKA pathway is and signaling molecule or substance upstream or downstream or cAMP or PKA, or their collection. An activator of adenylyl cyclase is an activator of a cAMP-PKA pathway, such as forskolin, although forskolin also results in signaling via cAMP-EPAC-PI3K pathway in A549 cells. An agonist for Gs-coupled receptors can also be an activator of cAMP-PKA pathway.

108. β2-Adrenergic Receptor Agonist

A β2-adrenergic receptor agonist is any molecule or substance which acts as an agonist at the β2-adrenergic receptor. For example, β2-adrenergic receptor agonists can be activators of Gs mediated signaling, such as epinephrine (Epi).

109. GPR109A

GPR109A is a high affinity receptor for nicotinic acid (niacin) (Wise, A., et al. (2003). “Molecular identification of high and low affinity receptors for nicotinic acid”. J. Biological Chemistry 278: 9869-9874; Soga, T., et al. (2003). “Molecular identification of nicotinic acid receptor”. Biochemical and Biophysical Research Communications 303: 364-369) and is a member of the nicotinic acid receptor family of G protein-coupled receptors (the other identified member being GPR109B). GPR109A is a Gi/Go protein-coupled receptor with high affinity for nicotinic acid. An agonist of GPR109A can be an activator of Gi-mediated signaling, such as nicotinic acid.

110. EGFRs

Epidermal growth factor receptors (EGFR) are a family of four receptor tyrosine kinases, including EGFR (ErbB-1), HER2/c-neu (ErbB-2), Her 3 (ErbB-3) and Her 4 (ErbB-4). Mutations affecting EGFR expression or activity could result in cancer. A431 cells endogenously express primarily EGFR, while HT29 cells express both EGFR and HER2. The EGFR receptor in A431 cells can activate at least PI3K pathway, PKC pathway, and MAPK pathway, through, for example, EGF.

111. HepG2 Cell Line

Hep G2 (ATCC No. HB-8065) is a human hepatocellular carcinoma cell line, and a perpetual cell line which was derived from the liver tissue of a 15 year old Caucasian American male with a well differentiated hepatocellular carcinoma. These cells are epithelial in morphology, have a model chromosome number of 55 and are not tumorigenic in nude mice. The cells secrete a variety of major plasma proteins; e.g., albumin, transferrin and the acute phase proteins fibrinogen, alpha 2-macroglobulin, alpha 1-antitrypsin, transferrin and plasminogen. The cells will respond to stimulation with human growth hormone.

HepG2 cells are a suitable in vitro model system for the study of polarized human hepatocytes. An other well-characterized polarized hepatocyte cell lines includes the rat hepatoma-derived hybrid cell line WIF-B. With the proper culture conditions, HepG2 cells display robust morphological and functional differentiation with a controlable formation of apical and basolateral cell surface domains that resemble the bile canalicular (BC) and sinusoidal domains, respectively, in vivo (see summary in http://www.ATCC.org).

112. HT29 Cell Line

The HT29 cell line comes from a human, Caucasian, colon, adenocarcinoma, grade II, epithelial cancer cell line. The ATCC catalog code is HTB-38. HT29 cell line secrets molecules including secretory component of IgA, carcinoembryonic antigen (CEA), transforming growth factor beta binding protein, and mucin during culture (see summary in http://www.ATCC.org).

113. A549 Cell Line

A549 cell line is carcinomic human alveolar basal epithelial cells. The ATCC catalog code is CCL-185. A549 is a hypotriploid human cell line initiated in 1972 through explant culture of lung carcinomatous tissue from a 58-year-old Caucasian male (see summary in http://www.ATCC.org).

114. A431 Cell Line

A431 cells are a model cell line used in biomedical research. More specifically, they are used in studies of the cell cycle and cancer-associated cell signaling pathways since they express abnormally high levels of the Epidermal growth factor receptor (EGFR). As such they are often used as a positive control for EGFR expression. They contain no functional p53, a potent tumor suppressor gene, and so are highly sensitive to mitogenic stimuli. A431 cells were established from an epidermoid carcinoma in the vulva of an 85 year old female patient.

Epidermal growth factor (EGF) stimulation of A431 cells induces rapid tyrosine phosphorylation of intracellular signaling proteins which control cellular processes such as growth, proliferation and apoptosis. At low (picomolar) concentrations EGF promotes cell growth of A431 cells whereas at higher (nanomolar) concentrations it inhibits growth by causing the cells to terminally differentiate. Treatment of A431 cells with bradykinin reduces basal and EGF-induced EGFR phosphorylation. Treatment with sertoli cell secreted growth factor (SCSGF) strongly induces cell proliferation. Stimulation of A431 cells with phorbol esters induces expression of interleukin 1-related protein IL1H.

115. Activator of the PKC and MAPK Pathways

An activator of the PKC or MAPK pathways is any molecule or substance that activates the PKC or MAPK pathways.

116. Activator of IGF1R Mediated Signaling

An activator of the IGF mediated signaling is any molecule or substance that activates signaling upstream or downstream from the IGF1R, such as an agonist for IGF1R, such as IGF1.

117. Activator of a hERG Pathway

An activator of the hERG pathway is any molecule or substance that activates the hERG pathway, such as an activator of hERG, such as mallotoxin.

118. Activator of Gq and Gi-Mediated Signaling and EGFR Transactivation

An activator of Gq and Gi-mediated signaling and EGFR transactivation is any molecule or substances that activates Gq and Gi-mediated signaling and EGFR transactivation, such as an agonist for the NTS1/NTS3, such as neurotensin.

119. Rho Kinase Inhibitor

A Rho Kinase (ROCK) inhibitor is any molecule or substance which has been determined to inhibit Rho Kinase, such as Y-27632, ROCK kinase inhibitor III Rockout, Rho kinase inhibitor IV, H89, and H8.

### I. Examples

### 1. Example 1

### Dose Dependent Responses of Two Well Known PI3K Inhibitors LY294002 and Wortmannin

i. Materials and Methods (for Examples 1 to 8)

a. Materials

Epinephrine, nicotinic acid, Y-27632, pinacidil, poly (I:C), forskolin, and histamine were purchased from Sigma Chemical Co. (St. Louis, Mo.). Mallotoxin, PI-103, LY303511, resveratrol, LY294002, quercetin, wortmannin, HA1077, H-8, and Phorbol 12-myristate 13-acetate (PMA) were obtained from BioMol International Inc (Plymouth Meeting, Pa.). Epidermal growth factor (EGF), neurotensin, insulin-like growth factor 1 (IGF-1), SLIGKV-amide was obtained from BaChem Americas Inc. (Torrance, Calif.). ODN2006 was obtained from Imgenex (San Diego, Calif. 92121). Rho kinase inhibitor III Rockout, Rho kinase inhibitor IV and CDK1/2 inhibitor III, PI3Kγ inhibitor 5-quinoxalin-6-ylmethylene-thiazolindine-2,4-dione, PI3Kβ inhibitor II TGX-221 ((+/−)-7-Methyl-2-(morpholin-4-yl)-9-(1-phenylaminoethyl)-pyridol[1,2-a]-pyrimidin-4-one), DNA PK Inhibitor II LY293646 (2-(Morpholin-4-yl)-benzo[h]chromen-4-one), DNA PK inhibitor III IC86621 (1-(2-Hydroxy-4-morpholin-4-yl-phenyl)ethanone) were obtained from EMD Biosciences (Gibbstown, N.J.). Epic® 384 biosensor microplates cell culture compatible were obtained from Corning Inc. (Corning, N.Y.).

b. Cell Culture

All cell lines were obtained from American Type Cell Culture (Manassas, Va.). The cell culture medium was as follows: (1) Dulbecco's modified Eagle's medium (DMEM) supplemented with 10% fetal bovine serum (FBS), 4.5 g/liter glucose, 2 mM glutamine, and antibiotics for human epidermoid carcinoma A431, human lung carcinoma A549 and human hepatocellular carcinoma HepG2; (2) McCoy's 5a Medium Modified supplemented with 10% FBS, 4.5 g/liter glucose, 2 mM glutamine, and antibiotics for human colorectal adenocarcinoma HT29.

Cells were typically grown using ˜1 to 2×104 cells per well at passage 3 to 15 suspended in 50 μl of the corresponding culture medium in the biosensor microplate, and were cultured at 37° C. under air/5% CO₂ for ˜1 day. Except for A431 which underwent one day culture followed by one starvation in serum free medium, all other cells were directly assayed without starvation. The confluency for all cells at the time of assays was ˜95% to 100%.

c. Optical Biosensor System and Cell Assays

Epic® beta version wavelength interrogation system (Corning Inc., Corning, N.Y.) was used for whole cell sensing. This system consists of a temperature-control unit, an optical detection unit, and an on-board liquid handling unit with robotics. The detection unit is centered on integrated fiber optics, and enables kinetic measures of cellular responses with a time interval of ˜15 sec.

The RWG biosensor is capable of detecting minute changes in local index of refraction near the sensor surface. Since the local index of refraction within a cell is a function of density and its distribution of biomass (e.g., proteins, molecular complexes), the biosensor exploits its evanescent wave to non-invasively detect ligand-induced dynamic mass redistribution in native cells. The evanescent wave extends into the cells and exponentially decays over distance, leading to a characteristic sensing volume of ˜150 nm, implying that any optical response mediated through the receptor activation only represents an average over the portion of the cell that the evanescent wave is sampling. The aggregation of many cellular events downstream the receptor activation determines the kinetics and amplitudes of a ligand-induced DMR.

For biosensor cellular assays, molecule solutions were made by diluting the stored concentrated solutions with the HBSS (1× Hanks balanced salt solution, plus 20 mM Hepes, pH 7.1), and transferred into a 384 well polypropylene molecule storage plate to prepare a molecule source plate. Both molecule and marker source plates were made separately when a two-step assay was performed. In parallel, the cells were washed twice with the HBSS and maintained in 30 μl of the HBSS to prepare a cell assay plate. Both the cell assay plate and the molecule and marker source plate(s) were then incubated in the hotel of the reader system. After ˜1 hr of incubation the baseline wavelengths of all biosensors in the cell assay microplate were recorded and normalized to zero. Afterwards, a 2 to 10 minute continuous recording was carried out to establish a baseline, and to ensure that the cells reached a steady state. Cellular responses were then triggered by pipetting 10 μl of the marker solutions into the cell assay plate using the on-board liquid handler.

To study the influence of molecules on a marker-induced response, a second stimulation with the marker at a fixed dose (typically at EC80 or EC100) was applied. The resonant wavelengths of all biosensors in the microplate were normalized again to establish a second baseline, right before the second stimulation. The two stimulations were usually separated by ˜1 hr.

All studies were carried out at a controlled temperature (28° C.). At least two independent sets of experiments, each with at least three replicates, were performed. The assay coefficient of variation was found to be <10%.

ii. Results

The PI3K and Ras oncoproteins are activated in many major tumor types and control linked signaling pathways. K-Ras activating mutants cause excessive activation of its downstream factors including Raf and PI3K. Since the human lung carcinoma cell line A549 expresses an activating mutant of K-Ras and thus exhibits deregulated PI3K pathway activity, this indicated that label-free biosensors can directly detect the impact of PI3K inhibitors acting on A549 cells. Two well known PI3K inhibitors LY294002 and wortmannin were examined. Both PI3K inhibitors belong to the first generation of PI3K inhibitors, and are widely known and extensively studied. Both have co-crystal structures with PI3Kγ and have played important roles in elucidating the signaling mechanism of the PI3K pathway, as well as exploiting the exact functions of each isoform of PI3Ks. Wortmannin is a fungal metabolite, first isolated in 1957 and fully recognized in terms of structure in 1974. It was described as a potent inhibitor of respiratory burst in neutrophils and monocytes in 1987 and determined to be a potent PI3K inhibitor in 1993. Wortmannin irreversibly inhibits PI3Ks including PI3Kα via covalent interaction with the catalytic lysine residue within the ATP binding site of PI3K, at low nanomolar concentrations. LY294002, a synthetic pan-PI3K inhibitor, was first described as a competitive PI3K inhibitor in 1994 with micromolar IC50 value towards class I PI3Ks.

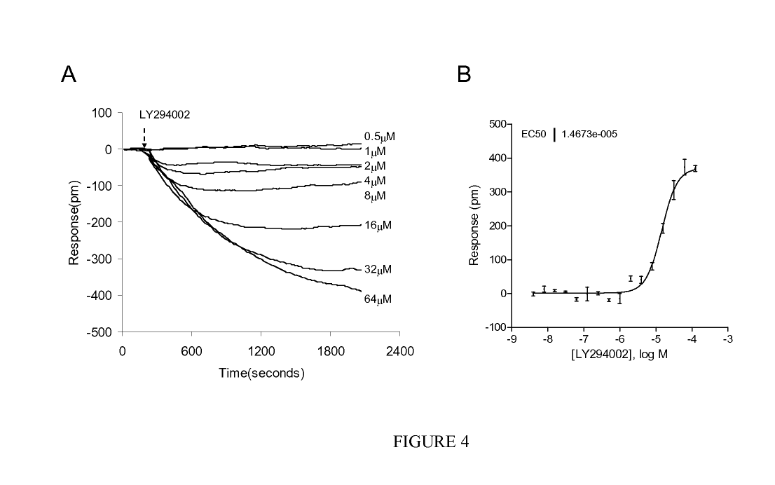

As shown in FIG. 4, the pan-PI3K inhibitor LY294002 caused a dose-dependent and saturable DMR signal in A549 cells, as measured using the RWG biosensor Epic®system. Based on the amplitudes of the N-DMR signals, the LY294002 N-DMR signal was found to be saturable, leading to an EC50 of 14.7±1.5 micromolar, with a maximal amplitude of ˜400 picometers (n=4).

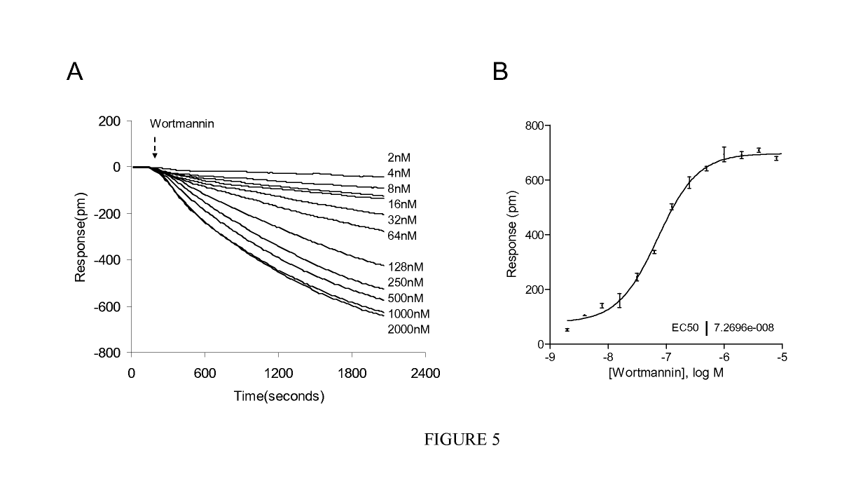

Similarly, the irreversible PI3K inhibitor wortmannin also led to similar DMR signals in a dose-dependent manner, yielding an EC50 of 72.7±3.4 nanomolar with a maximal response of ˜700 picometers (n=4) (FIG. 5). In contrast, both PI3K inhibitors did not produce significant biosensor signals in A431 cells (FIG. 7A and data not shown), a cell line that does not have deregulated PI3K pathway although EGF receptor is over-expressed in A431 cells. These results indicate that the label-free biosensors are capable of directly detecting the impacts of PI3K inhibitors in live as well as characterizing any PI3K pathway deregulated cells such as A549 cells.

### 2. Example 2

### Modulation Profiles of a Panel of Markers by PI3K Inhibitors

LY294002 and quercetin

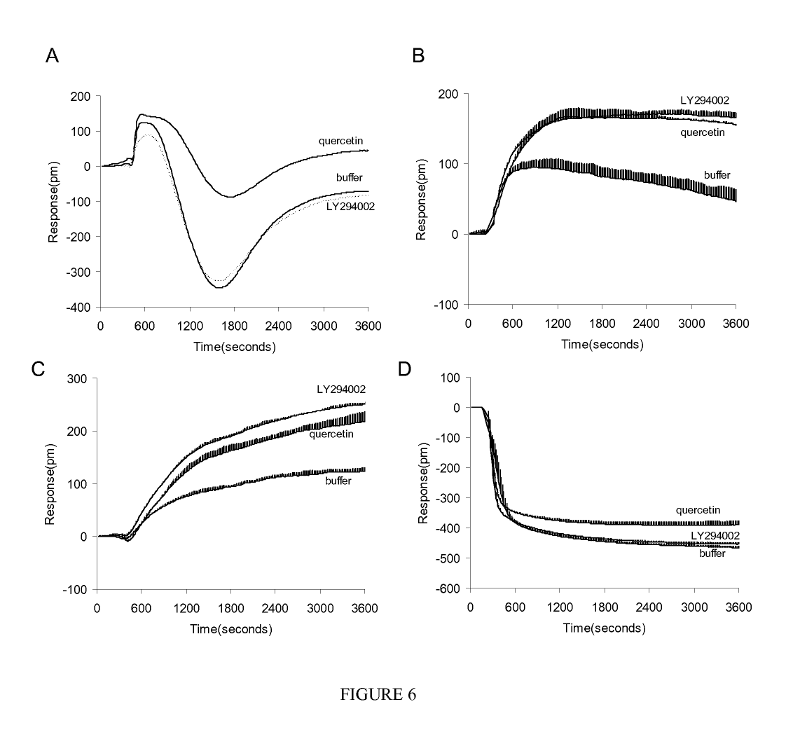

Label-free biosensors measure an integrated cellular response upon stimulation, and the resultant biosensor cellular signals contain contributions from downstream signaling pathways via the cellular target. The PI3K pathway is involved in signaling mediated through a wide array of targets including receptor tyrosine kinases and G protein-coupled receptors (GPCRs), thus PI3K inhibitors are able to modulate the biosensor signals induced by these targets. Since the expression patterns and basal activity of PI3K isoforms could be quite different among different types of cells, the modulation profiles of panels of markers are of interest across different types of cells by PI3K inhibitors. Accordingly two cell lines: A549 and A431 cells were chosen. As shown in FIG. 6A, the two known PI3K inhibitors LY294002 and quercetin modulated the EGF-induced DMR signals differently in quiescent A431 cells. Quecertin is known to have polypharmacology, including inhibitory activity on PI3K, agonism activity on GPR35, etc. The pan-PI3K inhibitor LY294002 attenuated the initial P-DMR event and the subsequent N-DMR event of the EGF DMR signal in A431 cells, while quercetin only attenuated the N-DMR event of the EGF DMR signal.

However, both compounds potentiated the P-DMR signals of both histamine and forskolin in A549 cells (FIG. 6B and FIG. 6C, respectively). On the other hand, LY294002 had little impact on the DMR signal of HepG2 cells in response to stimulation with TLR9 agonist ODN2006, while quercetin slightly attenuated the ODN2006 DMR signal (FIG. 6D).

EGF is the natural agonist of EGF receptor. Forskolin is an activator of adenylyl cyclases, whose activation leads to cAMP-PKA pathway. Histamine is a natural agonist of endogenous histamine receptors in A549 cells. ODN2006 is a synthetic agonist of Toll-like receptor 9 (TLR9). TLR9 is an intracellular receptor. EGF receptor signaling is well-known to lead to the activation of PI3K pathway in A431 cells. However, both forskolin and histamine mediated signaling pathways in A549 were poorly characterized in literature. Here both chemicals triggered a signaling sensitive to PI3K activity. Similarly, little is known for the presence and functions of TLR9 in HepG2 cells. Here TLR9 is expressed and functioned in HepG2 cells and can be detected robustly by the optical biosensor.

Taken together, the results indicated that although the known PI3K inhibitors LY294002 and quercetin display different polypharmacology, both chemicals gave rise to similar phenotypic pharmacology in the PI3K pathway deregulated cell line A549, and the modulation profiles, particularly the modulation patterns against the panel of markers, can be used to compare and characterize distinct PI3K inhibitors.

### 3. Example 3

### Characterization of a Series of PI3K Inhibitors Using DMR Indices

Optical biosensors such as RWG biosensor measure dynamic mass redistribution (DMR) signals of cells upon stimulation. A DMR index contains the primary DMR profile or signal of a chemical acting on a panel of live cells, and the modulation index of the chemical against panels of markers across the panel of cells, a panel of markers for each cell line. A panel of markers were chosen whose biosensor signals in the given cell types contain contributions from PI3K pathway to characterize the PIK inhibitors. These markers are the adenylyl cyclase activator forskolin and the histamine receptor agonist histamine in A549 cells, the EGF receptor agonist EGF in A431 cells. The ATP-sensitive potassium ion channel activator pinacidil in A549 was also included as a negative control for a PI3K inhibitor, but a positive control for ROCK inhibitors. The pinacidil DMR signal in A549 is insensitive to PI3K inhibitors, but can be fully attenuated by ROCK inhibitors such as Y27632. To test specificity of such DMR indexing approach for characterizing PI3K inhibitors, over a 1000 compound library was screened containing some known PI3K inhibitors.

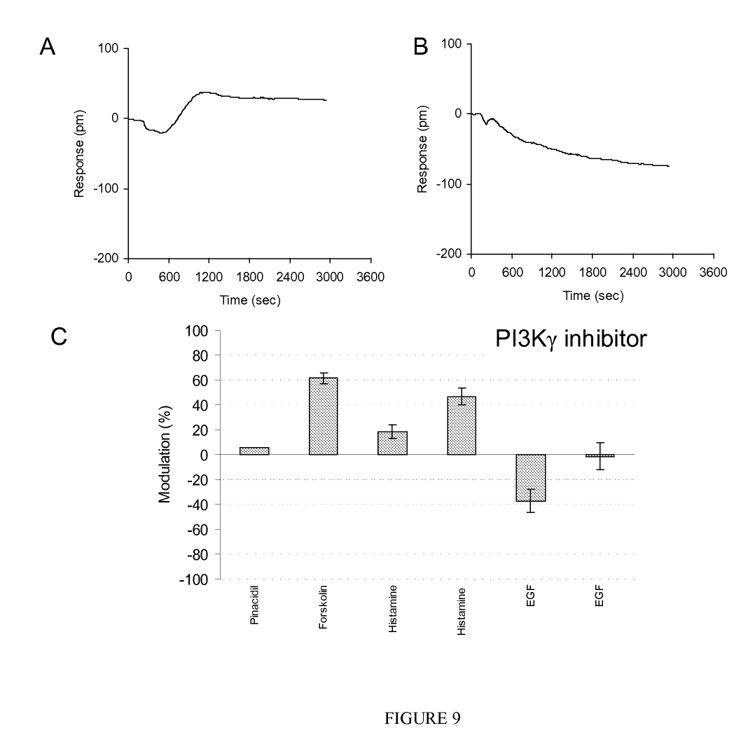

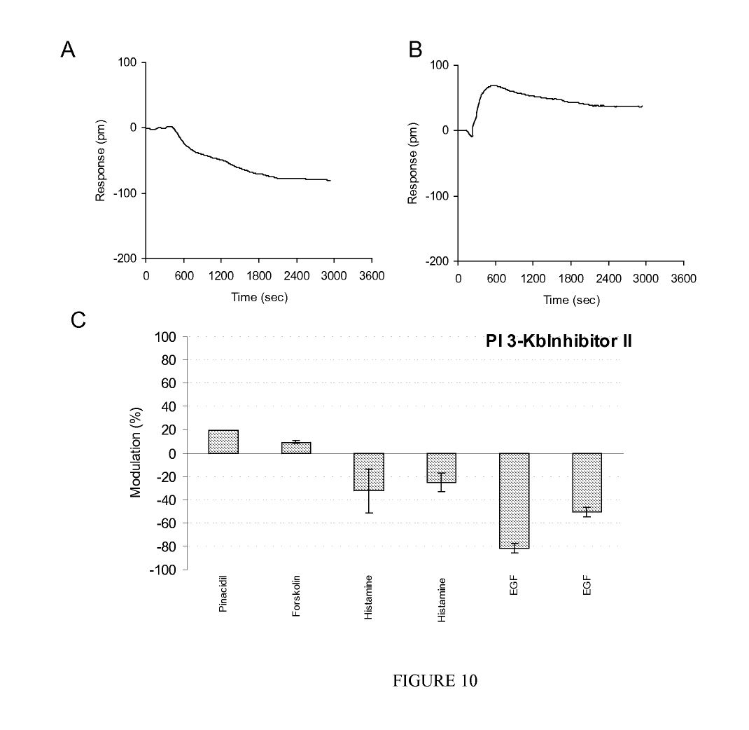

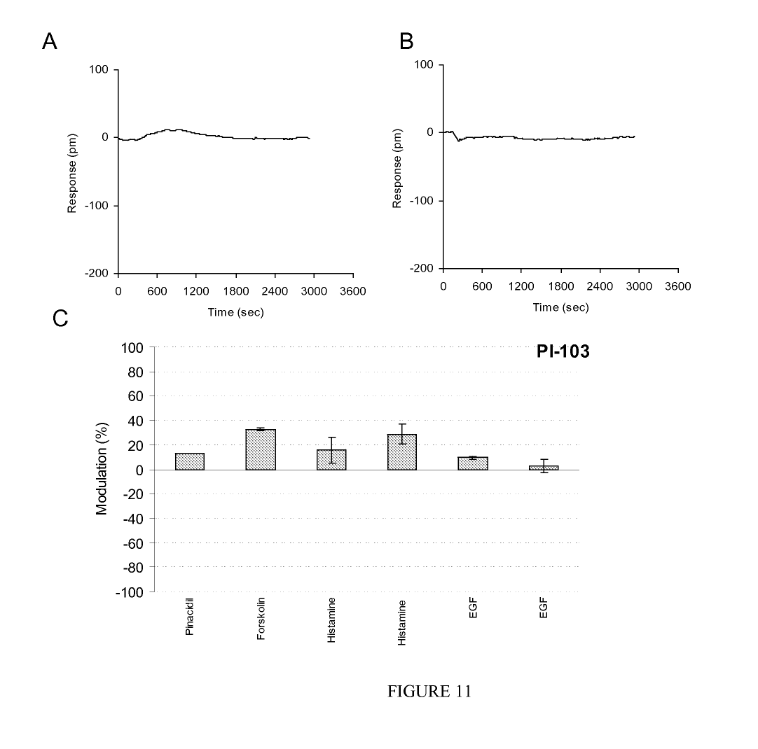

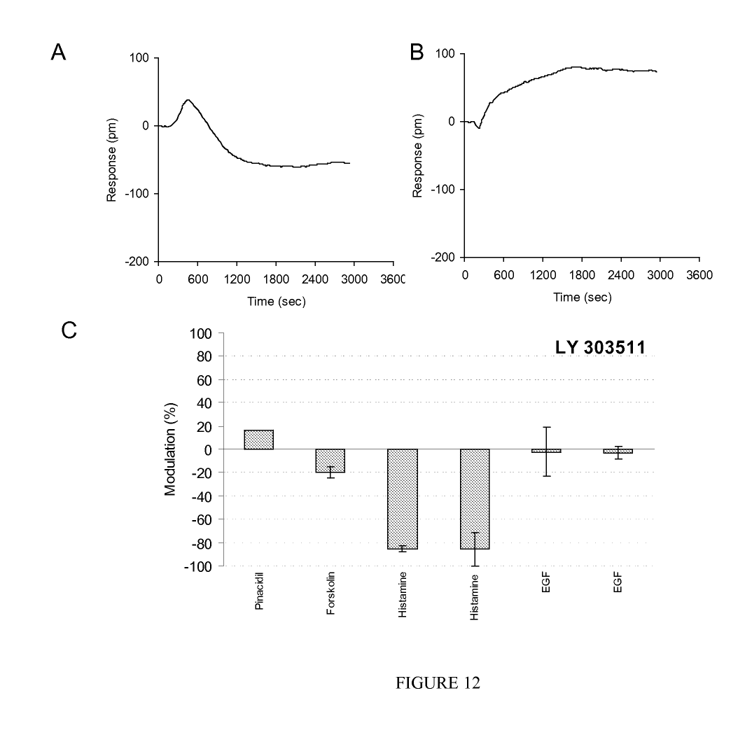

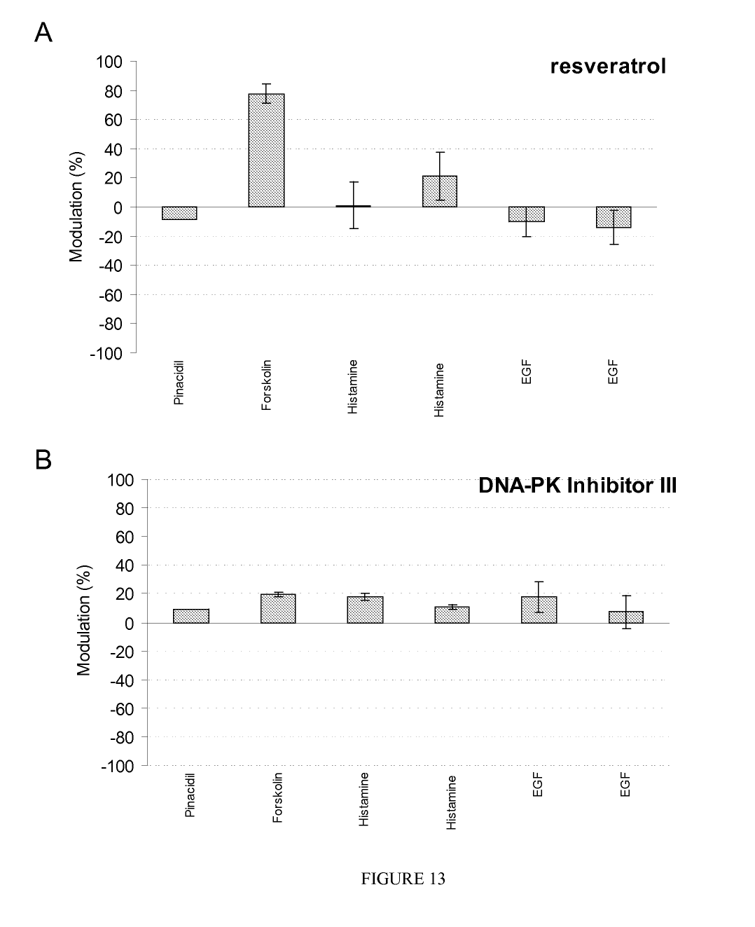

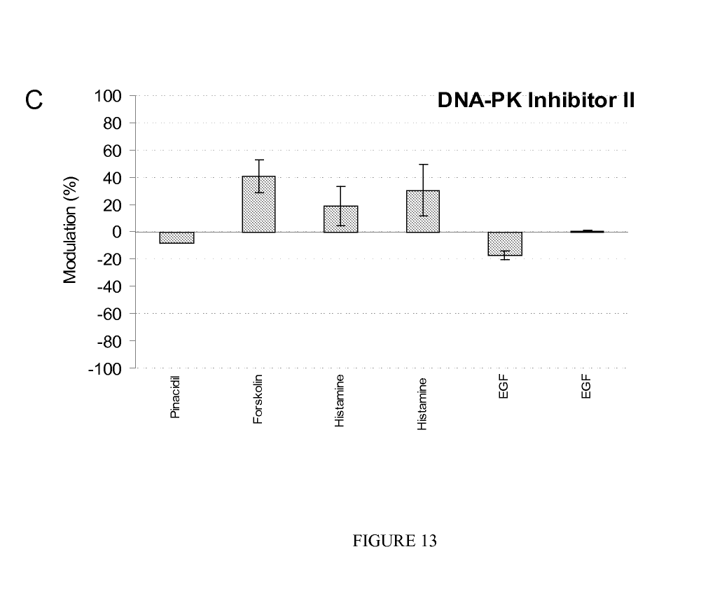

Results showed that the most well-characterized pan-PI3K inhibitor LY294002 led to little DMR signal in A431 cells (FIG. 7A), a N-DMR signal in A549 cells (FIG. 7B), and a unique modulation index against the panels of markers (i.e., little modulation of the pinacidil response in A549, potentiation of the forskolin response in A549 as well as both the early and late DMR signals of the histamine response in A549, and attenuation of both the P-DMR and N-DMR events of the EGF response in A431) (FIG. 7C). Based on the similarity of chemical DMR modulation index to the PI3K inhibitor LY294002, ˜20 hits were identified. Among them, except for the PI3Kβ inhibitor TGX-221 (FIG. 10), all known PI3K inhibitors in the library had been identified, including quercetin (FIG. 8), the PI3KKγ inhibitor II 5-quinoxalin-6-ylmethylene-thiazolindine-2,4-dione (FIG. 9), PI-103 (FIG. 11), resveratrol (FIG. 13A), DNA-PK inhibitor III (FIG. 13B) and DNA-PK inhibitor II (FIG. 13C), all of which the DMR modulation indexes are similar (i.e., significant potentiation in the forskolin response and the histamine responses by these inhibitors, and small attenuation of the P-DMR event of the EGF response in A431 cells, little or not modulation of the pinacidil response in A549 cells). However, there is significant difference in the primary DMR signals in both A431 and A549 cells among these inhibitors, possibly due to the differences in their potency as well as isoform selectivity. Also significant differences exist in the pattern and percentages of modulation by difference PI3K inhibitors, which also indicate isoform selectivity. For example, Taking the late P-DMR event of the histamine response in A549 cells, the pan PI3K inhibitor LY294002 led to the largest potentiation, while the PI3Kγ inhibitor gave rise to moderate potentiation, and the PI3Kβ inhibitor II led to slightly suppression, reflecting that PI3Kγ, but not PI3Kβ, is the major component of the Gq-coupled H1 receptor mediated signaling in A549 cells. The expression of PI3Kβ isoform in A549 is unknown in literature. In A431 cells, PI3Kβ isoform is known to be the downstream of EGFR signaling. As expected, the inhibition of PI3Kβ isoform by this inhibitor significantly attenuated the P-DMR event of the EGF signal in A431 cells (FIG. 10C). The LY294002 negative control compound LY303511 led to completely distinct type of DMR index (FIG. 12).

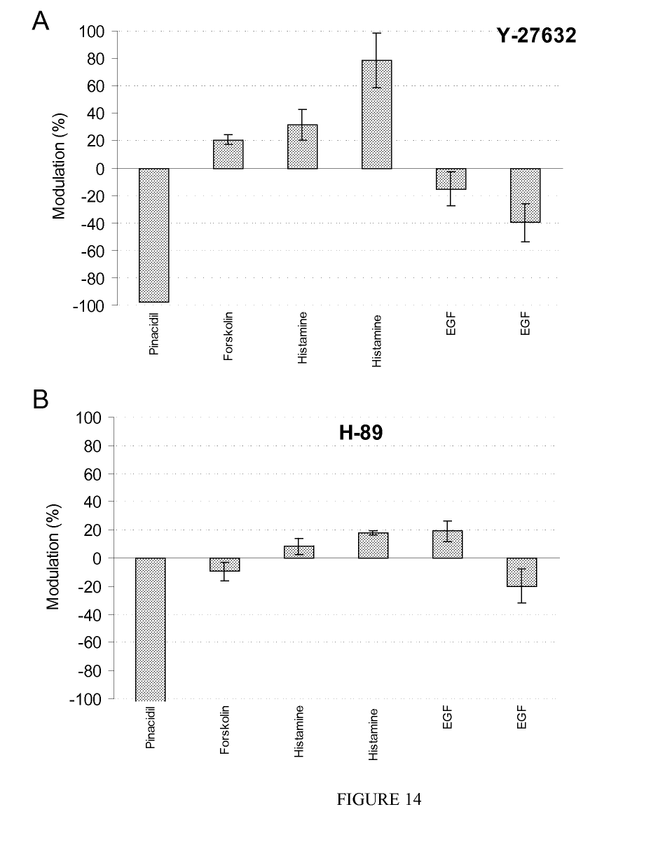

Interestingly, a series of ROCK inhibitors including Y-27632 (FIG. 14A), Rho kinase inhibitor III Rockout, Rho kinase inhibitor IV, and a series of PKA inhibitor including H-89 (FIG. 14B), H-8, H-9, H-7, HA-1004, and HA-1077 also led to somewhat similar DMR modulation index, except for the complete or significant attenuation of the pinacidil response in A549 cells by these ROCK inhibitors (FIG. 14 and data not shown). The ROCK pathway is tightly linked to PI3K pathway. Importantly, the modulation profiles of the pinacidil signal by distinct chemicals can be used as a differentiating factor to separate ROCK inhibitors from PI3K inhibitors, since the pinacidil response in A549 is insensitive to PI3K inhibitors, but can be completely attenuated by ROCK inhibitors.

### 4. Example 4

### High Resolution Characterization of a Series of PI3K Inhibitors Using DMR Indices

It is well known that many kinase inhibitors often display polypharmacology (i.e., many kinase inhibitors can modulate the activity of multi-targets in a cell). In addition, drugs including kinase inhibitor often display cell- or tissue-dependent phenotypic pharmacology, due to many cellular factors including post-transcriptional modifications, compartmentalization of signaling cascades, alteration of key gene products in the pathway, receptor oligomerization and constitutive receptor activity. As a result, a ROCK inhibitor may not behave as a ROCK inhibitor in a cell line that Rho kinases are down-regulated; similarly, other kinase inhibitors such as protein kinase A inhibitors H-89, HA-1004, H-8, H-9, HA-1077, and H-7 may exhibit a ROCK inhibitor phenotypic pharmacology in a cell line that PI3K pathway is amplified (e.g., through down-regulation of a negative feedback loop such as the phosphatase PTEN) or up-regulated. Thus, the primary modes of action of a drug or drug candidate molecule, including ROCK and PI3K inhibitors, are a sum of at least several types of pharmacology—polypharmacology, phenotypic pharmacology and systems (integrative) pharmacology. Thus, in order to correctly detect the phenotypic pharmacology of a PI3K inhibitor that is indeed inhibiting PI3K activity in live cells, one can use a panel of cells as well as panels of markers using the biosensor cellular assays at high resolution. Accordingly four different cell lines were chosen: A549, A431, HT29 and HepG2 cells. A panel of markers were also chosen for A549 cells; and these markers are pinacidil (an ATP-sensitive potassium ion channel opener), poly(IC) (an agonist for toll-like receptor subtype 3, TLR3), PMA (a small molecule activator for protein kinase C), SLIGKV-amide (an agonist for endogenous protease activated receptor subtype 2), forskolin (an activator for adenylyl cyclase), and histamine (an agonist for endogenous histamine receptors). All of these markers led to dose-dependent and saturable biosensor signals, but with distinct dynamics and characteristics (data not shown). The concentrations of these markers closer to EC100 were chosen for generating the biosensor modulation indexes of any molecules, particularly ROCK inhibitors and PI3K inhibitors. Each of these markers also leads to a wide array of cell signaling.

Using Epic® cellular assays in conjunction with chemical biology and cell biology approach, the pathway(s) and network interaction(s) were identified that accounts for each marker-induced DMR signal in A549 cells. The main observations are summarized below. Poly(I:C) is an agonist for endogenous Toll-like receptor(s) in A549, whose activation leads to IKK pathway and AKT pathway. SLIGKV-amide is an agonist for endogenous protease activated receptor subtype 2 (PAR2) in A549, whose activation leads to Gq-, Gi- and G12/13-mediated signaling. Pinacidil is an opener for endogenous ATP-sensitive potassium (KATP) ion channel in A549, whose activation leads to Rho- and JAK-mediated signaling. PMA is an activator for protein kinase C (PKC), whose activation leads to PKC pathway and degranulation. Histamine is a natural agonist for endogenous histamine receptors (dominantly H1R, and possibly H3R), whose activation leads to dual signaling, possibly through Gq and G1 mediated signaling in A549. Forskolin is an activator for adenylyl cyclases, whose activation leads to cAMP-PKA pathway in A549.

Four markers were chosen for A431 cells: the endogenous β2-adrenergic receptor agonist epinephrine (Epi), the endogenous GPR109A agonist nicotine acid (NA), the EGFR agonist EGF, and the endogenous histamine receptor 1 (H1R) agonist histamine (His). Epinephrine triggered a Gs mediated signaling, nicotinic acid led to a Gi-mediated signaling, histamine led to a Gq-mediated signaling, while EGF triggered at least three signaling pathways (PI3K pathway, PKC pathway and MAPK pathway) (data not shown). For each marker, the EC80 or EC100 concentration was chosen to determine the modulation index of a compound.

Four markers were also chosen for HT29 cells: the endogenous EGF receptor agonist EGF, the endogenous IGF1 receptor agonist IGF1, the endogenous hERG activator mallotoxin, and the endogenous NTS1/NTS3 agonist neurotensin (NT). In HT29 cells, EGF triggered both PKC and MAPK pathways, while IGF1 triggered IGF1R mediated signaling, mallotoxin triggered MAPK pathway, and NT led to both Gq and Gi-mediated signaling as well as EGFR transactivation (data not shown). For each marker, the EC80 or EC100 concentration was chosen to determine the modulation index of a compound.

ODN2006, an agonist for endogenous TLR9 receptor in HepG2, was chosen as a marker, since the ODN2006 biosensor signal in HepG2 cells was found to be potentiated by the pretreatment of cells with the known Rho kinase inhibitor Y-27632 (data not shown), but not PI3K inhibitors (FIG. 6).

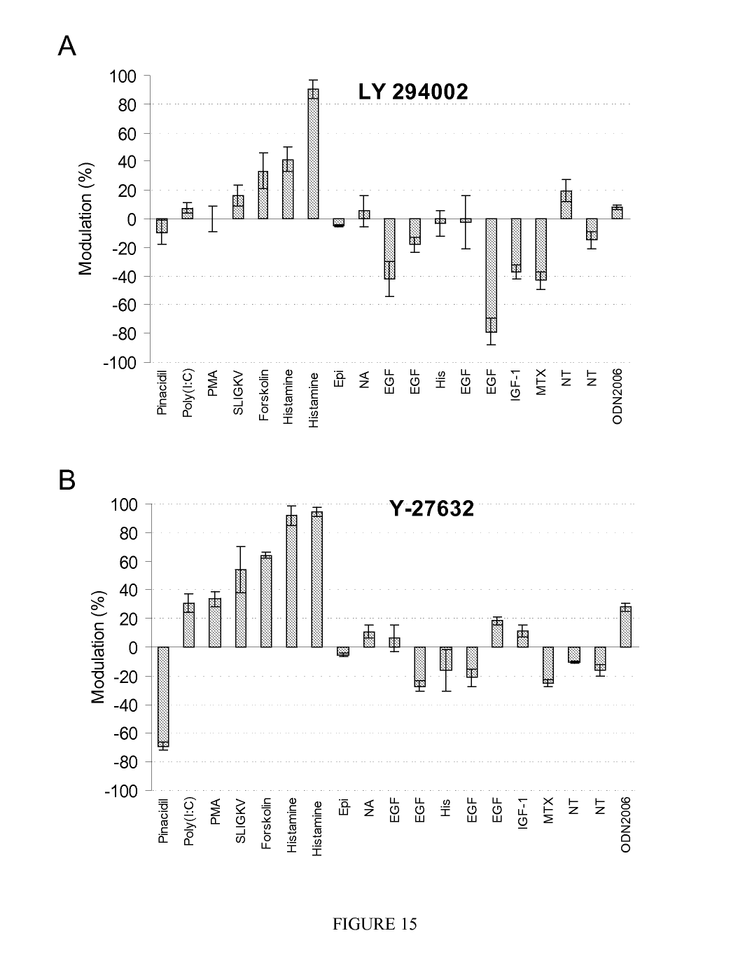

FIG. 15 compares the DMR indices of the ROCK inhibitor Y17631 and the PI3K inhibitor LY-294002. Results showed that such high resolution characterizes both inhibitors in details regarding to their phenotypic pharmacology, and polypharmacology. Such approach can be used to easily separate two classes of inhibitors, which target two distinct but closely linked kinases in the PI3K pathways.

### 5. Example 5

### Screening Compounds that are Able to Attenuate the Y-27632 Induced Biosensor Response in Live Cells

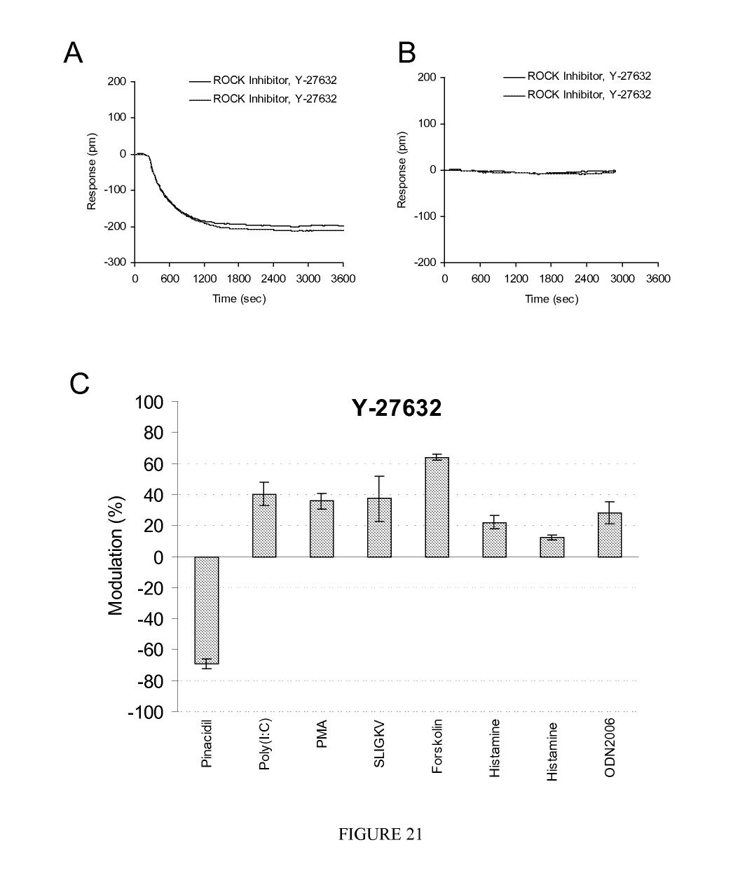

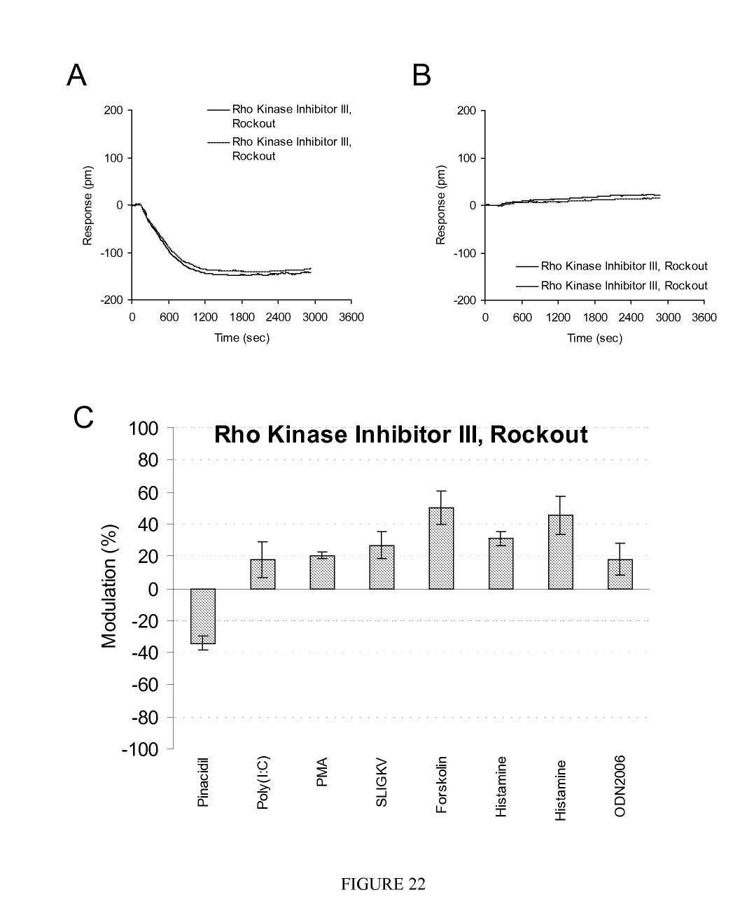

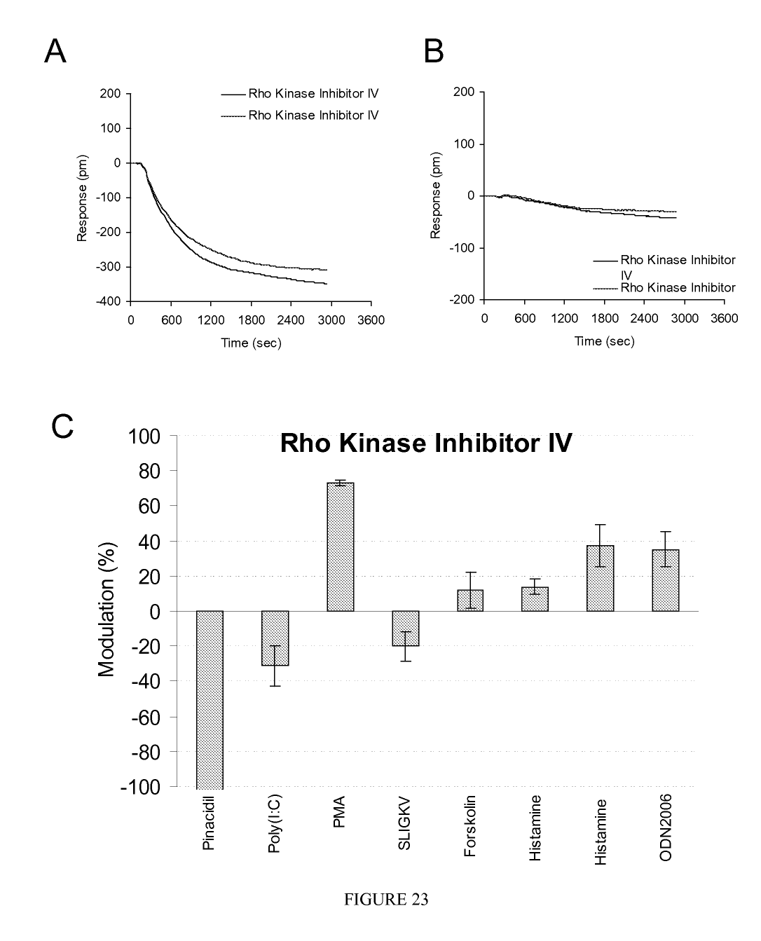

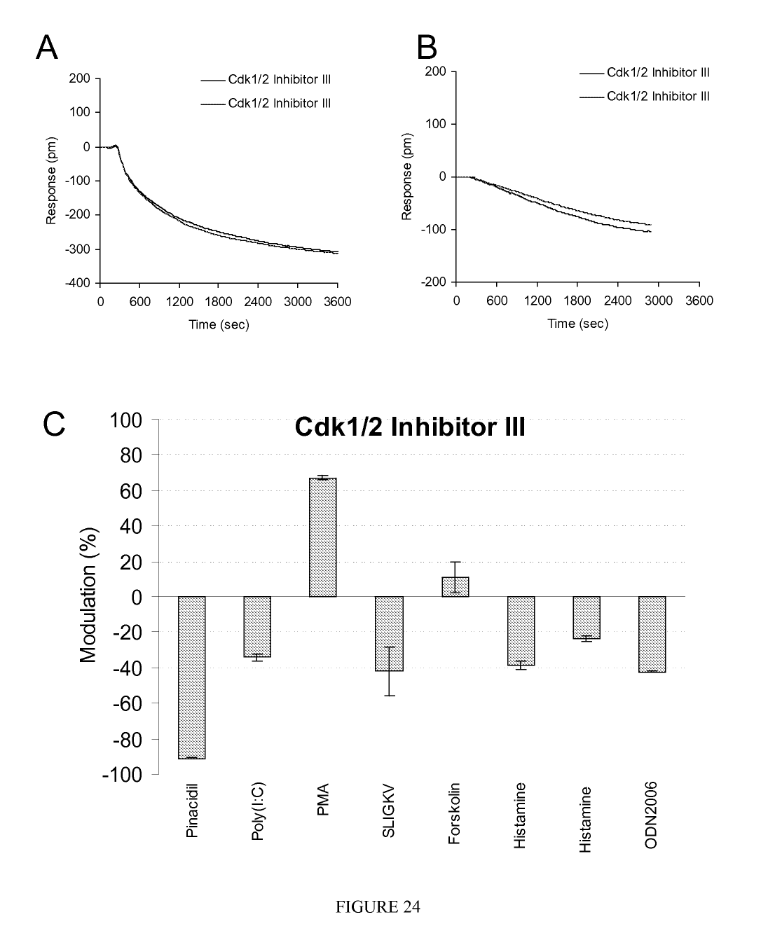

Since ROCKs regulate cell morphology inhibition of basal ROCK activity by ROCK inhibitor could lead to detectable biosensor signals. To demonstrate four cell lines were chosen: human skin cancer cell line A431, human lung cancer cell line A549, human colon cancer cell line HT-29 and human live cell line HepG2. Stimulation of cells with a well-established ROCK inhibitor Y-27632 led to detectable, and dose-dependent biosensor signals using Corning® Epic® RWG biosensor in either A431, A549 or HT-29 cells, but not in HepG2 cells (FIG. 21, FIG. 22, FIG. 23 and data not shown). Thus, A431, A549, or HT-29 cells are considered to be ROCK inhibitor responsive cells.

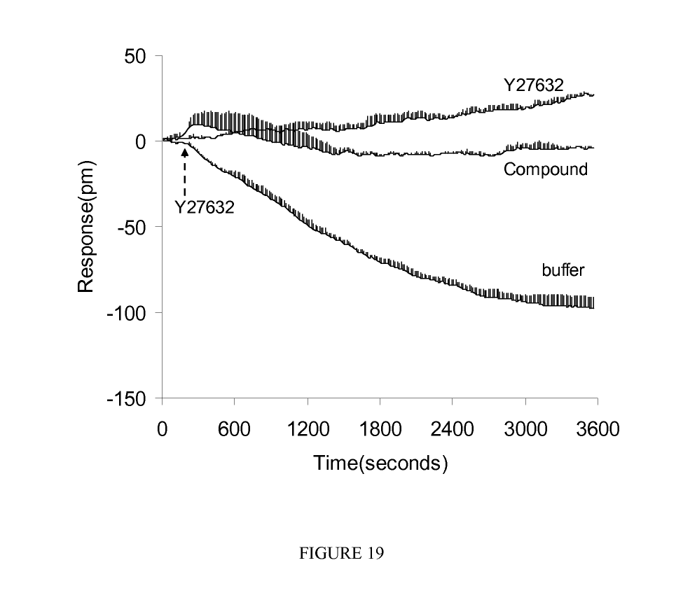

The PI3K pathway is often amplified in all types of lung cancers (e.g., squamous cells, adenocarcinoma lung cells, small lung cancer cell, non-small-lung cancer cells), and the ROCK pathway is tightly linked to the PI3K pathway, therefore A549 cells were used to screen ROCK inhibitors. Accordingly, the A549 cells were individually pretreated with compounds (each at 10 micromolar) for a specific period of time (typically about 1 hour), and then stimulated with the ROCK inhibitor Y-27632 at EC100 (10 micromolar). As shown in FIG. 19, the preceding treatment with Y27632 significantly caused the cells desensitize to the second Y-27632 stimulation. Similarly, ROCK kinase inhibitor III Rockout (“compound” in FIG. 19) also led to similar desensitization response in A549 cells. By choosing appropriate end-point measurements, such approach can be applied to high throughput screening potential ROCK inhibitors.

### 6. Example 6

### Screening Compounds that are Able to Attenuate a ROCK Pathway Sensitive Marker-Induced Biosensor Response in a Live Cell

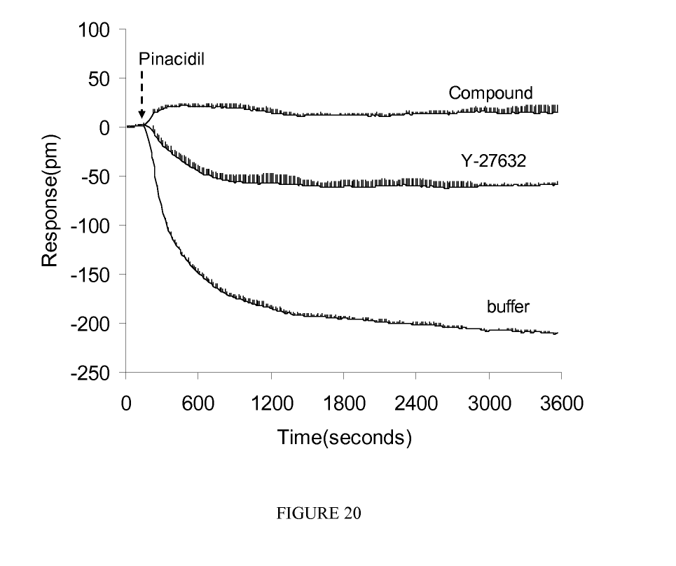

Many external stimuli can lead to the activation of ROCK pathway. Label-free biosensor cellular assays are able to detect the downstream signaling including ROCK pathway mediated through the external stimuli, thus, these external stimuli can be used as markers to screen ROCK inhibitors. One of the examples is shown in FIG. 20. Pinacidil is an ATP-sensitive potassium ion channel opener. Stimulation of A549 cells with pinacidil led to a dose-dependent and saturable biosensor signal with an EC50 of ˜5 micromolar (FIG. 20; and data not shown). The known ROCK inhibitor Y027632 can dose-dependently attenuate the pinacidil response; and Y27632 at 10 micromolar almost completely attenuated the pinacidil response. Screening the 80 kinase inhibitor library led to identify a cluster of compounds that led to similar modulation profiles. These compounds include two other known ROCK inhibitors, Rho kinase inhibitor III Rockout and Rho kinase inhibitor IV, and a family of known PKA inhibitors including H-89, H-7, H-8, HA-1004, H-9, and HA1077. Literature mining results also suggest that these H-series PKA inhibitors are also known to be ROCK inhibitors. Taken together, these results suggest that the disclosed methods can be used to determine the phenotypic pharmacology of known kinase inhibitors in a specific cell type.

### 7. Example 7

### Characterizing the Phenotypic Pharmacology and Polypharmacology of ROCK Inhibitors Using Panels of Cells/Markers with the Biosensor Cellular Assays

It is well known that many kinas inhibitors often display polypharmacology (i.e., many kinase inhibitors can modulate the activity of multi-targets in a cell). In addition, drugs including kinase inhibitors often display cell- or tissue-dependent phenotypic pharmacology, due to the many cellular factors including post-transcriptional modifications, compartmentalization of signaling cascades, alteration of key gene products in the pathway, receptor oligomerization, and constitutive receptor activity. As a result, a ROCK inhibitor cannot behave as a ROCK inhibitor in a cell line that Rho kinases are down-regulated; similarly, other kinase inhibitors such as protein kinase A inhibitors H-89, HA-1004, H-8, H-9, HA-1077, and H-7 could exhibit a ROCK inhibitor phenotypic pharmacology in a cell line that a Rho kinase pathway is amplified (e.g., through down-regulation of a negative feedback loop such as the phosphatase PTEN) or up-regulated. Thus, the primary modes of action of a drug or drug candidate molecule, including ROCK inhibitors, are a sum of at least several types of pharmacology—polypharmacology, phenotypic pharmacology and systems (integrative) pharmacology. To correctly detect the phenotypic pharmacology of a ROCK inhibitor that acts via the inhibition of ROCK activity in live cell, one needs a panel of cells as well as panels of markers using the biosensor cellular assays. Thus, a ROCK inhibitor responsive cell line, A549, and a ROCK inhibitor unresponsive cell line, HepG2, were chosen as model systems. A panel of markers for A549 cells was also chosen: pinacidil (an ATP-sensitive potassium ion channel opener), poly(IC) (an agonist for toll-like receptor subtype 3, TLR3), PMA (a small molecule activator for protein kinase C), SLIGKV-amide (an agonist for endogenous protease activated receptor subtype 2), forskolin (an activator for adenylyl cyclase), and histamine (an agonist for endogenous histamine receptors). All of these markers led to dose-dependent and saturable biosensor signals, but with distinct dynamics and characteristics (data not shown). The concentrations closer to EC100 were chosen for these markers for generating the biosensor modulation indexes of any molecule, particularly ROCK inhibitors.

As shown in FIG. 21, Y-27632 at 10 micromolar concentration led to a characteristic negative DMR signal, but led to a net-zero DMR signal in hepG2 cells. The pretreatment of A549 cells with Y-27632 attenuated the pinacidil response, but potentiated the poly(IC) response, the PMA response, the SLIGKV response, the forskolin response, as well as both the early and later response of the histamine DMR signal. Similarly, Y-27632 pretreatment also potentiated the ODN2006 DMR signal in HepG2 cells.

Using the panel of cells/markers, we systematically characterized over 500 compounds including ROCK inhibitor Y-27632. A subset of molecules in the library led to biosensor indices including the primary profiles in corresponding cells and the modulation index against the panel of markers that are similar to the Y-27632 biosensor index. These molecules were Rho kinase inhibitor III Rockout, Rho kinase inhibitor IV, and a series of PKA inhibitor including H-89, H-8, H-9, H-7, HA-1004, and HA-1077. Examples for the two other Rho kinase inhibitors were shown in FIG. 22, and FIG. 23. In contrast. Also shown in FIG. 23 is the DMR index of the CDK1/2 inhibitor III, which also led to a negative DMR signal in A549 cells. However, this kinase inhibitor exhibited a quite distinct biosensor index, particularly it triggered a small decaying signal in HepG2 cells, and attenuated the biosensor signals of most of markers in the panel (FIG. 23).

### J. References

1. WO2006108183. Fang, Y., Ferrie, A. M., Fontaine, N. M., Yuen, P. K. and Lahiri, J. “Optical biosensors and cells”
2. U.S. application Ser. No. 12/623,693. Fang, Y., Ferrie, A. M., Lahiri, J., and Tran, E. “Methods for Characterizing Molecules”, Filed Nov. 23, 2009
3. U.S. application Ser. No. 12/623,708. Fang, Y., Ferrie, A. M., Lahiri, J., and Tran, E. “Methods of creating an index”, filed Nov. 23, 2009.
4. Okudela, K. et al. Am. J. Pathology, 2004, 164, 91-100.
5. Downward, J. Nature Medicine 2008, 14, 1315-1316.
6. Yap, T. A., et al. Curr. Opinion Pharmacol. 2008, 8, 393-412.
7. Engelman, J. A. Nature Reviews Cancer, 2009, 9, 550-562.
8. Serra, V., et al., Cancer Res. 2008, 68, 8022-8030.

1. A method of assaying a molecule comprising the steps:
a. culturing a deregulated PI3K pathway cell line on a surface, wherein the surface can be used in a label free biosensor analysis,
b. incubating the cell line with the molecule producing a molecule treated cell line,
c. analyzing the molecule treated cell line with a label free biosensor producing a molecule data output,
d. comparing the molecule data output to a known PI3K inhibitor data output in the same cell line, producing a molecule-PI3K inhibitor comparison.

2. The method of claim 1, wherein the known PI3K inhibitor data output was produced by incubating the PI3K inhibitor with the cultured deregulated PI3K cell line and analyzing the incubated cell line with a label free biosensor producing a PI3K inhibitor data output.

3. The method of claim 1, further comprising identifying a potential PI3K inhibitor when the comparison indicates that the molecule data output and the PI3K inhibitor data output are similar.

4. The method of claim 1, wherein the deregulated PI3K pathway cell line is selected from a K-Ras activating mutant cell line, a HER2 overexpressed cell line, a PTEN loss-of-function cell line, or a PI3KCA activating mutant cell line.

5. The method of claim 4, wherein the K-Ras activating mutant cell line is A549 cell line, or H820 cell line.

6. The method of claim 4, wherein the PTEN loss-of-function cell line is A4 cell line, A7 cell line, human giant-cell lung cancer cell line 95C, human giant-cell lung cancer cell line 95D, or breast cancer cell line MDA-468.

7. The method of claim 4, wherein the HER2 overexpressed cell line is BT474, or SKBR3.

8. The method of claim 4, wherein the PI3KC activating mutant cell line is MCF-7, or T47D.

9. The method of claim 1, wherein the known PI3K inhibitor comprises LY294002, or wortmannin.

10. The method of claim 1, further comprising incubating the molecule with a normal PI3K pathway cell line, analyzing the molecule-treated cell line with a label free biosensor producing a second molecule data output, and comparing the second molecule data output to a PI3K inhibitor data output produced in the same cell line.

11. The method of claim 10, wherein the cell line overexpresses EGF receptor.

12. The method of claim 11, wherein the cell line is the A431 cell line.

13. A method of assaying a molecule comprising the steps:
a. culturing a deregulated PI3K pathway cell line on a surface, wherein the surface can be used in a label free biosensor analysis,
b. incubating the cell line with a marker and the molecule producing an incubating cell line,
c. analyzing the incubating cell line with a label free biosensor producing a marker-molecule data output
d. comparing the marker-molecule data output to a marker-PI3K inhibitor data output in the same cell line, producing a marker-molecule/marker-PI3K inhibitor comparison.

14. The method of claim 13, wherein the marker-PI3K inhibitor data output was produced by incubating the marker and PI3K inhibitor with a cultured deregulated PI3K cell line and analyzing the incubated cell line with a label free biosensor producing a marker-PI3K inhibitor data output.

15. The method of claim 13, further comprising identifying a potential PI3K inhibitor when the comparison indicates that the marker-molecule data output and the marker-PI3K inhibitor data output are similar.

16. The method of claim 13, further comprising incubating the marker and molecule with a normal PI3K pathway cell line, analyzing the marker-marker treated cell line with a label free biosensor producing a second marker-molecule data output, and comparing the second marker-molecule data output to a marker-PI3K inhibitor data output produced in the same cell line.

17. The method of claim 15, further comprising incubating the marker and molecule with a Toll-like receptor cell line, analyzing the Toll-like receptor incubating cell line with a label free biosensor producing a third marker-molecule data output, and comparing the third marker-molecule data output to a marker-PI3K inhibitor data output produced in the same cell line.

18.-27. (canceled)

28. The method of claim 13, further comprising assaying the molecule for ROCK pathway activity.

29. The method of claim 28, wherein the step of assaying comprises,
a. culturing a ROCK inhibitor responsive cell line on a surface, wherein the surface can be used in a label free biosensor analysis,
b. incubating the cell line with the molecule producing an incubating cell line,
c. analyzing the incubating cell line with a label free biosensor producing a molecule data output,
d. comparing the molecule data output to a known ROCK inhibitor data output in the same cell, producing a molecule-ROCK inhibitor comparison.

30.-48. (canceled)

49. A method of assaying a molecule comprising the steps:
a. culturing four different cell lines on a surface, wherein the surface can be used in a label free biosensor analysis, wherein the four cell lines are A431, A549, HT29, and HepG2
b. incubating each cell line with the molecule producing four incubating cell lines,
c. analyzing each incubating cell line with a label free biosensor producing a molecule data output for each incubating cell line,
d. comparing the molecule data output to a ROCK inhibitor data output in the same cell line, producing a molecule-ROCK inhibitor comparison for each cell line.

50.-52. (canceled)
